(19) United States
(12) Patent Application Publication
(10) Pub. No.: US 2012/0259237 A1
(43) Pub. Date: Oct. 11, 2012

(54) FLUID INJECTION SYSTEM COMPRISING A MOTION TRANSFER CABLE

(75) Inventor: Michael Axelrod, Roswell, GA (US)

(73) Assignee: CARTICEPT MEDICAL, INC., Alpharetta, GA (US)

(21) Appl. No.: 13/438,691

(22) Filed: Apr. 3, 2012

Related U.S. Application Data
(60) Provisional application No. 61/472,075, filed on Apr. 5, 2011.
(60) Provisional application No. 61/551,818, filed on Oct. 26, 2011.

(51) Int. Cl. A61M 5/20; A61B 8/00

(52) U.S. Cl. 600/506; 604/154; 600/439

(57) ABSTRACT

An injection system for transferring a volume of fluid from a syringe into a patient comprises a syringe assembly having a syringe adapter configured to securely receive a pre-filled syringe and a plunger assembly configured to be selectively moved relative to the syringe adapter. The injection system further comprises a fluid delivery module comprising at least one motor to move an actuator in at least one direction. In some embodiments, the injection system additionally comprises a motion transfer cable. In one embodiment, one end of the motion transfer cable is mechanically coupled to the actuator of the fluid delivery module, and the other end is mechanically coupled to the plunger assembly of the syringe assembly. In some embodiments, movement of the actuator by the motor is transferred through the motion transfer cable to the plunger assembly, resulting in the plunger assembly moving a desired distance relative to the pre-filled syringe.

### RELATED APPLICATIONS AND INCORPORATION BY REFERENCE

This application claims priority benefit of U.S. Provisional Application Nos. 61/472,075, filed Apr. 5, 2011, and 61/551,818, filed Oct. 26, 2011, the entireties of which are hereby incorporated by reference herein and made a part of the present specification. The entireties of U.S. patent application Ser. No. 12/340,595, filed Dec. 19, 2008 and issued on Aug. 30, 2011 as U.S. Pat. No. 8,007,487, and U.S. patent application Ser. No. 12/823,004, filed Jun. 24, 2010 and published on Jan. 27, 2011 as U.S. Publication No. 2011/0021905 are also hereby incorporated by reference herein and made a part of the present specification.

### BACKGROUND

1. Field

This application relates generally to injection and/or aspiration devices, systems and methods, and more specifically, to devices, systems and methods of delivering pharmaceuticals, fluids and/or other substances to or near a joint or another anatomical location of a patient.

2. Description of the Related Art

Physicians, clinicians and/or other medical personnel often need to deliver a volume of medication, other fluid and/or other material to (or aspirate fluid from) an anatomical location, such as, for example, a joint (e.g., toe, knee, wrist, shoulder, ankle, finger, spine, etc.). Accordingly, a needle can be inserted through a patient's skin and into the targeted location. A syringe or other fluid source that is in fluid communication with the needle can then be used to deliver the desired volume or other dosage of fluid and/or other material to the targeted joint or other anatomical location. Some fluids and/or other medicaments that are delivered into the anatomy can be relatively viscous and/or dense. Further, such fluids and/or other medicaments are often transferred through a small needle into a joint or other portion of the anatomy. The relatively small diameter of needles used in such procedures and the relatively high backpressure encountered during the delivery of fluids and/or other medicaments into joints or other portions of the anatomy present challenges to the physician or other clinician performing such injection procedures.

Current injection practice generally involves palpation by the physician of a bony prominence on the patient's anatomy to serve as a “landmark” to guide the injection into the targeted location. The injection is completed by advancing the needle, which is typically connected to a disposable glass or plastic syringe, into the target area. The syringe plunger is then advanced to deliver the fluid. In many cases, current treatment methods do not offer precise or accurate delivery and do not offer a convenient way of delivering fluids and/or other materials, especially those with relatively high densities or viscosities against relatively high backpressures.

### SUMMARY

According to some embodiments, an injection system for transferring a volume of fluid from a syringe (e.g., a pre-filled syringe or other standard or non-standard container) into a patient comprises a syringe assembly comprising a syringe adapter configured to securely receive a syringe and a plunger assembly configured to be selectively moved relative to the syringe adapter, at least one motor assembly configured to impart a force on and move an actuator in at least one direction and a motion transfer cable comprising a first end and a second end and an inner core extending between said first end and said second end. In some embodiments, the first end of the motion transfer cable is mechanically coupled to the actuator, and the second end of the motion transfer cable is mechanically coupled to the plunger assembly of the syringe assembly. In one embodiment, movement of the actuator by the motor is transferred through the motion transfer cable to the plunger assembly, resulting in the plunger assembly moving a desired distance relative to the syringe secured to the syringe assembly. In some embodiments, fluid exiting the syringe as a result of movement by the actuator, the motion transfer cable and the plunger assembly passes through a distal outlet of the syringe. In one embodiment, the syringe assembly is configured to be grasped and manipulated by a clinician during an injection procedure.

According to some embodiments, a method of injecting a fluid stored within a syringe (e.g., a pre-filled syringe or other standard or non-standard container) into a patient comprises securing a syringe to a syringe adapter of a syringe assembly of an injection system, wherein the injection system further comprises a motor assembly and a motion transfer cable, the motion transfer cable comprising an inner core, wherein the motion transfer cable mechanically couples the syringe assembly to the motor assembly. The method further comprises coupling a first terminal of the motion transfer cable to an actuator of the motor assembly, wherein at least one motor of the motor assembly is configured to selectively move the actuator. In some embodiments, the method additionally comprises coupling the second terminal of the motion transfer cable to a plunger assembly of the syringe assembly, wherein the plunger assembly is configured to selectively move within an interior of the pre-filled syringe in order to discharge fluid from an outlet of the pre-filled syringe. In some embodiments, the method further comprises moving the actuator using the motor assembly in response to an injection instruction, wherein moving the actuator causes a corresponding linear motion in the first and second terminals and the inner core of the motion transfer cable extending between said first and second terminals. In one embodiment, movement of the second terminal toward the pre-filled syringe urges the plunger assembly into the interior of the pre-filled syringe and transfers a volume of fluid through the outlet of the pre-filled syringe. In some embodiments, fluid exiting the outlet of the pre-filled syringe passes through a needle assembly removably secured to a distal end of the pre-filled syringe and into a target anatomical location of a patient. In some embodiments, the syringe assembly is configured to be grasped and manipulated by a clinician during an injection procedure.

According to some embodiments, an injection system for transferring a volume of fluid from a pre-filled syringe into a patient comprises a syringe assembly comprising a syringe adapter configured to securely receive a pre-filled syringe and a plunger assembly configured to be selectively moved relative to the syringe adapter, at least one motor assembly configured to impart a force on and move an actuator in at least one direction and a motion transfer cable comprising a first end and a second end and an inner core extending between said first end and said second end. According to some embodiments, the first end of the motion transfer cable is mechanically coupled to actuator of the at least one motor assembly, and the second end of the motion transfer cable is mechanically coupled to the plunger assembly of the syringe assembly. In one embodiment, movement of the actuator by the motor is transferred through the motion transfer cable to the plunger assembly, resulting in the plunger assembly moving a desired distance relative to the pre-filled syringe positioned within the syringe assembly. In some embodiments, fluid exiting the pre-filled syringe as a result of movement by the actuator, the motion transfer cable and the plunger assembly passes through a distal outlet of the pre-filled syringe and through a needle attached thereto. In some embodiments, the syringe assembly is configured to be grasped and manipulated by a clinician during an injection procedure. According to some embodiments, the motor assembly is operatively coupled to fluid delivery module. In some embodiments, the motor assembly comprises a coupling, fitting or other feature configured to removably receive a connector operatively coupled to the motion transfer cable. In some embodiments, the coupling, fitting or other features and the connector comprise a quick-connect connection.

According to some embodiments, a filling system for at least partially filling a syringe or other container with one or more fluids comprises a manifold comprising at least one inlet port and at least one fluid channel in fluid communication with the at least one inlet port, an outlet port and a valve or fluid selector generally positioned between the fluid channel and the outlet port. In some embodiments, the valve is configured to move between one of a plurality of positions. In one embodiment, the valve is configured to selectively place the fluid channel in fluid communication with the outlet port when the valve is in a first position. In some embodiments, the inlet port is configured to receive a fluid container (e.g., a vial or other container). In some embodiments, such a vial or other container is supplied directly or indirectly by a medicament manufacturer or supplier. In some embodiments, such a vial or container can comprise various sizes, shapes and/or configurations. In some embodiments, the outlet port is configured to be placed in fluid communication with a syringe or other container. In one embodiment, the fluid channel is not in fluid communication with the outlet port when the valve is in a second position. In some embodiments, the outlet port is configured to be removably coupled to a syringe or other container comprising one or more openings. In some embodiments, the syringe or other container that is coupled to the outlet port is part of a motion transfer cable injection system, in accordance with one or more embodiments disclosed herein. In some embodiments, the syringe or other container can be filled by retracting the plunger of the syringe using, at least in part, a motion transfer cable system.

According to some embodiments, the inlet port of the manifold comprises a first inlet port and a second inlet port. Further, the fluid channel comprises a first fluid channel and a second fluid channel. In some embodiments, the first fluid channel is in fluid communication with the first inlet port, and the second fluid channel is in fluid communication with the second inlet port. In some embodiments, the valve is configured to be moved between the first position, the second position and a third position, wherein the first inlet port is in fluid communication with the outlet port via the first fluid channel when the valve is in the first position, the second inlet port is in fluid communication with the outlet port via the second fluid channel when the valve is in the third position and neither the first inlet port nor the second inlet port is in fluid communication with the outlet port when the valve is in the second position.

According to some embodiments, the inlet port is configured to removably attach to a vial connector that is configured to receive vials or other containers having various sizes and shapes. In one embodiment, the valve is rotatable between the plurality of positions. In some embodiments, the valve is configured to move between the plurality of positions manually. In other embodiments, the valve is configured to move between the plurality of positions using an actuator or other movable device (e.g., a fluid delivery module, a stepper motor, another type of motor, solenoid, etc.). In some embodiments, the actuator comprises a motor or a solenoid. In some embodiments, the outlet port and/or one or more of the inlet port comprise a luer fitting and/or some other type of standard or non-standard quick-connect fitting. In one embodiment, the outlet port is configured to directly attach to the syringe or other container (e.g., via the corresponding luer connections of the outlet port and the syringe or other container). In some embodiments, the outlet port is configured to be in fluid communication with the syringe via a fluid conduit. In one embodiment, the inlet port comprises a first inlet port, a second inlet port and a second inlet port.

According to some embodiments, an injection system for transferring a volume of fluid from a pre-filled syringe into a patient comprises a syringe assembly having a syringe adapter configured to securely receive a pre-filled syringe and a plunger assembly configured to be selectively moved relative to the syringe adapter. The injection system further comprises a fluid delivery module comprising at least one motor configured to impart a force on and move an actuator in at least one direction (e.g., linearly, in two or more directions, etc.). In some embodiments, the injection system additionally comprises a motion transfer cable (e.g., a push/pull cable) comprising a first end (e.g., terminal) and a second end (e.g., terminal) and an inner core extending between the first and second ends. In one embodiment, the first end of the motion transfer cable is mechanically coupled to actuator of the fluid delivery module, and the second end of the motion transfer cable is mechanically coupled to the plunger assembly of the syringe assembly. In some embodiments, movement of the actuator by the motor is transferred through the motion transfer cable to the plunger assembly, resulting in the plunger assembly moving a desired distance relative to the pre-filled syringe positioned within the syringe assembly. In some embodiments, fluid exiting the pre-filled syringe as a result of movement by the actuator, the motion transfer cable and the plunger assembly passes through a distal outlet of the pre-filled syringe and through a needle attached thereto. In one embodiment, the syringe assembly is configured to be grasped and selectively manipulated by a clinician during an injection procedure.

In other embodiments, an injection system having a motion transfer cable is configured to transfer fluids to a syringe. For example, retracting of the motion transfer cable can create a negative pressure or vacuum within a syringe assembly to withdraw bodily or other fluids from a patient's anatomy. Such a configuration can be used, for instance, to drain excess fluid from a joint, an organ and/or the like.

According to some embodiments, the injection system is configured to receive instructions for moving the actuator and transferring a volume of fluid out of the pre-filled syringe. In one embodiment, the pre-filled syringe comprises hyaluronic acid, a platelet rich plasma (PRP), one or more other blood components, drugs, cells, liquid and non-liquid fluids and flowable materials, nanoparticles, cement, microbeads, therapeutics or diagnostic fluids, imaging fluids, lavage fluids, other endogenous or exogenous fluids or materials etc.) and/or any other substance or material. In some embodiments, the actuator is mechanically coupled to a cassette that is removably attached to the fluid delivery module. In one embodiment, the cassette comprises a riser coupled to the first end or terminal of the motion transfer cable and also coupled to the actuator of the fluid delivery module.

According to some embodiments, the motion transfer cable is generally flexible to permit the syringe assembly to be manipulated by a clinician during an injection procedure. In one embodiment, the motion transfer cable comprises at least one protective outer sheath (e.g., outer cases, outer conduit or sheath, etc.), wherein the inner core of the motion transfer cable is slidably movable relative to such a protective outer sheath. In some embodiments, the syringe adapter of the syringe assembly is configured to receive syringes of varying sizes, shapes and types. In one embodiment, the syringe adapter is configured to removably couple to a flange portion of a pre-filled syringe.

According to some embodiments, the injection system additionally comprises at least one controller for selectively adjusting a position of the actuator. In one embodiment, the controller comprises at least one button, dial, knob, switch, rollerball, softkey, rollerwheel and/or the like. In some embodiments, the controller comprises at least one button or softkey located on a display of the fluid delivery module. In some embodiments, the fluid delivery module comprises a display configured to provide status information of an injection procedure. In some embodiments, the fluid delivery module is in data communication with an imaging device (e.g., ultrasound device) configured to help locate a targeted anatomical location within the patient. In some embodiments, the motor of the fluid delivery module comprises a stepper motor, another type of motor and/or any other mechanically, pneumatically or hydraulically operated device.

According to some embodiments, the injection system further comprises a handpiece assembly, wherein an interior portion of the handpiece is sized, shaped and/or otherwise configured to receive and secure at least a portion of the syringe assembly (e.g., the barrel of a pre-filled syringe). In some embodiments, the handpiece assembly comprises a handle portion and an adapter portion, wherein the handle portion is configured to removably secure to the adapter portion (e.g., using corresponding tabs and/or recesses, other mating features or members, etc.). In one embodiment, the syringe assembly is configured to be positioned within an interior cavity of the adapter portion. In other embodiments, at least a portion of the handpiece assembly is configured to be disposable. In one embodiment, the syringe assembly is configured to secure to the adapter portion, wherein the syringe assembly and the adapter portion being configured to be discarded as a unitary structure after an injection procedure.

According to some embodiments, a method of transferring fluids to or from a syringe comprises securing a syringe to a syringe adapter of a syringe assembly of an injection system, wherein the injection system further comprises a motor and a motion transfer cable mechanically coupling the syringe assembly to the motor. The method further comprises securing a first terminal of the motion transfer cable to an actuator of the motor to selectively move said actuator. The method additionally includes securing the second terminal of the motion transfer cable to a plunger assembly of the syringe assembly, such that the plunger assembly is configured to move within an interior of the syringe in order to discharge fluid from an outlet of said syringe or to aspirate fluids from a patient into the syringe. In some embodiments, the method comprises moving the actuator using the at least one motor in response to an injection instruction; wherein moving the actuator causes a corresponding linear motion in first and second terminals and the inner core of the motion transfer cable extending between said first and second terminals. In one embodiment, movement of the second terminal toward the syringe urges the plunger assembly into the interior of the syringe and transfers a volume of fluid through the outlet of the syringe, and movement of the second terminal away the syringe urges the plunger assembly out of the interior of the syringe and transfers a volume of fluid into the syringe. In some embodiments, fluid exiting or entering the syringe passes through a needle assembly removably secured to a distal end of the syringe. In one embodiment, the syringe assembly is configured to be grasped and manipulated by a clinician during an injection procedure.

According to some embodiments, a method of injecting a fluid stored within a pre-filled syringe into a patient comprises securing a pre-filled syringe to a syringe adapter of a syringe assembly of an injection system, wherein, the injection system further comprises a fluid delivery module and a motion transfer cable mechanically coupling the syringe assembly to the fluid delivery module. The method additionally comprises securing a first terminal of the motion transfer cable to an actuator of the fluid delivery module, wherein the fluid delivery module includes at least one motor configured to selectively move the actuator. In some embodiments, the method further comprises securing the second terminal of the motion transfer cable to a plunger assembly of the syringe assembly, wherein the plunger assembly is configured to move within an interior of the pre-filled syringe in order to discharge fluid from an outlet of the pre-filled syringe.

The method further comprises moving the actuator using the motor in response to an injection instruction. In some embodiments, moving the actuator causes a corresponding linear motion in first and second terminals and the inner core of the motion transfer cable extending between the first and second terminals. In some embodiments, movement of the second terminal toward the pre-filled syringe urges the plunger assembly into the interior of the pre-filled syringe and transfers a volume of fluid through the outlet of the pre-filled syringe. In some embodiments, fluid exiting the outlet of the pre-filled syringe passes through a needle assembly removably secured to a distal end of the pre-filled syringe and into a target anatomical location of a patient. In some embodiments, the syringe assembly is configured to be grasped and manipulated by a clinician during an injection procedure.

### BRIEF DESCRIPTION OF THE DRAWINGS

These and other features, aspects and advantages of the present application are described with reference to drawings of certain embodiments, which are intended to illustrate, but not to limit, the various inventions disclosed herein. It is to be understood that the attached drawings are for the purpose of illustrating concepts and embodiments of the present application and may not be to scale.

FIG. 1 illustrates a partial perspective view of an injection system according to one embodiment;

FIG. 2 illustrates an exploded perspective view of an injection system according to another embodiment;

FIG. 3A illustrates a side view of one embodiment of a motion transfer cable configured for use with the injection system of FIGS. 1 and 2;

FIG. 3B illustrates a detailed side view and partial cross-sectional view of the motion transfer cable of FIG. 3A;

FIG. 3C schematically illustrates a side view of one embodiment of a motion transfer cable configured for use with the injection system of FIGS. 1 and 2;

FIGS. 4 and 5 illustrate detailed side views of the motion transfer cable of FIG. 3;

FIG. 6A illustrates one embodiment of a distal portion of the injection system of FIGS. 1 and 2;

FIG. 6B schematically illustrates the embodiment of the syringe assembly and the motion transfer cable of FIG. 6A;

FIG. 7 illustrates a perspective view of a cassette according to one embodiment; and

FIG. 8 illustrates an interior view of a cassette configured to be used with the injection system of FIGS. 1 and 2, according to one embodiment;

FIGS. 9 and 10 illustrate different views of a motor and accompanying components of one embodiment of a fluid delivery module configured for use with the injection system of FIGS. 1 and 2;

FIG. 11 illustrates one embodiment of a fluid delivery module of an injection system operatively coupled to an ultrasound device or other imaging system;

FIG. 12 illustrates another embodiment of a fluid delivery module of an injection system operatively coupled to an ultrasound device or other imaging system;

FIG. 13 illustrates a perspective view of a user simultaneously manipulating both an imaging wand and a syringe assembly of an injection system to treat a patient's foot according to one embodiment;

FIG. 14 illustrates a perspective view of a handpiece assembly configured to house a prefilled syringe according to one embodiment;

FIG. 15 illustrates an exploded perspective view of the handpiece assembly of FIG. 14;

FIG. 16 illustrates a perspective view of the disposable adapter portion of the handpiece assembly of FIG. 14 with a syringe assembly secured therein;

FIG. 17 illustrates the disposable adapter of FIG. 16 with the syringe assembly removed;

FIG. 18A schematically illustrates a partial perspective view of an injection system according to one embodiment;

FIG. 18B illustrates a partial detailed view of the injection system of FIG. 18A;

FIG. 19A illustrates a perspective view of a system for filling a syringe according to one embodiment;

FIG. 19B schematically illustrates another embodiment of a system for filling a syringe;

FIG. 20 illustrates a top perspective view of a base for use with the filling system of FIG. 19A;

FIG. 21A illustrates a front perspective view of a manifold for use with the filling system of FIG. 19A;

FIG. 21B illustrates a bottom perspective view of the manifold of FIG. 21A;

FIG. 22A illustrates a front perspective view of a valve or fluid selector for use with the filling system of FIG. 19A;

FIG. 22B illustrates a cross-sectional view of the valve of FIG. 22A; and

FIG. 23 illustrates a top view of the assembled base, manifold and valve of FIGS. 20-22B.

### DETAILED DESCRIPTION

The discussion and the figures illustrated and referenced herein describe various embodiments of an injection device and system, as well as methods related thereto. A number of these embodiments of injection systems, devices and methods are particularly well suited to accurately and easily transfer a volume of one or more fluids and/or other materials to (or from) or near (and/or from) an intra-articular or joint space, a bone, an organ, cavity or other location of the human anatomy (e.g., toe, foot, ankle, knee, hand, finger, etc.). Such devices, systems and methods are well-suited for treating various joint diseases, including, but not limited to, osteoarthritis, rheumatoid arthritis, other inflammatory diseases and/or other conditions. However, the various devices, systems, methods and other features of the embodiments disclosed herein may be utilized or applied to other types of apparatuses, systems, procedures and/or methods, including arrangements that have non-medical benefits or applications.

As discussed in greater detail herein, this application discloses devices, systems and methods of delivering (and/or withdrawing) fluids and/or other materials (e.g., medications, pharmaceutical compositions, drugs, cells, platelet-rich plasma (PRP), other plasma or blood components or formulations, hyaluronic acid, liquid and non-liquid fluids and flowable materials, nanoparticles, cement, microbeads, therapeutics or diagnostic fluids, imaging fluids, lavage fluids, other endogenous or exogenous fluids or materials etc.) to (and/or from) joints, organs, vessels and/or any other anatomical locations. According to some embodiments, the devices, systems and methods disclosed herein facilitate the delivery and/or aspiration of fluids and/or other materials to and/or from an intra-articular space (e.g., joint) or other anatomical location. Also disclosed are methods of accurately locating the target joint or other anatomical location prior to an injection or aspiration sequence. Such systems, devices and methods can be especially useful for the treatment of smaller joints, such as, for example, toes, thumbs, other fingers and/or the like that are highly innervated. In addition, such devices and methods can simplify the execution of related procedures by physicians and other medical personnel. According to some embodiments, the devices, systems and methods disclosed herein help a clinician or other user to locate targeted joints or other anatomical spaces for the subsequent accurate delivery of fluids and/or other materials thereto.

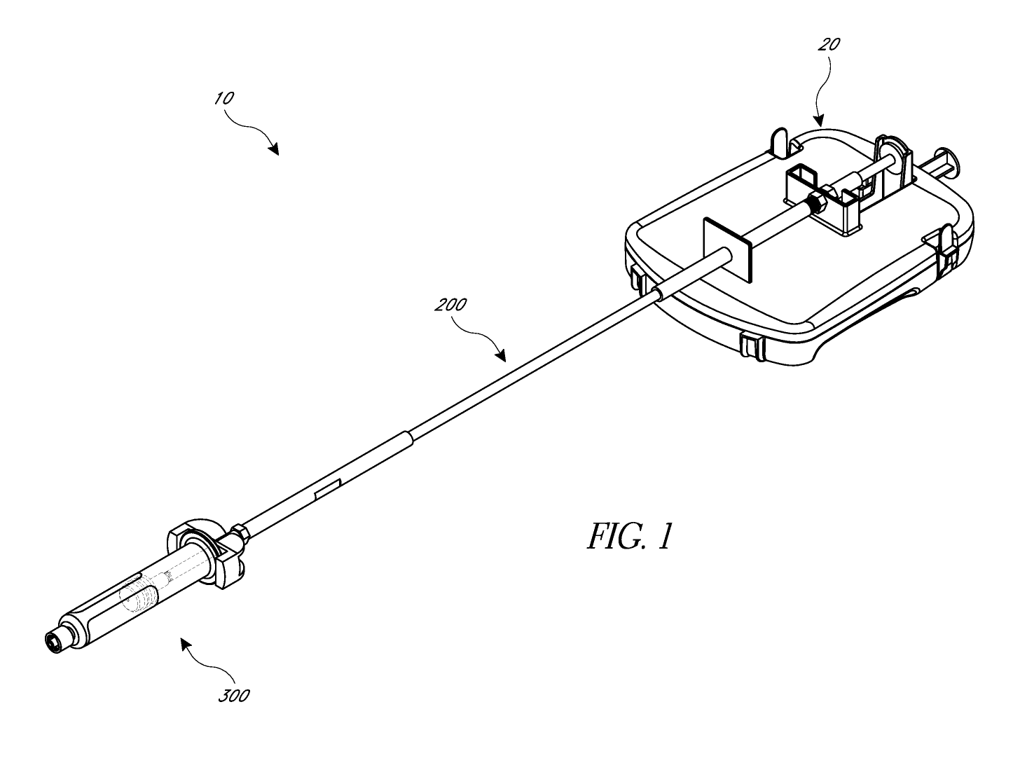

FIG. 1 illustrates a perspective view of certain components of an injection system 10 according to one embodiment. As shown, the injection system 10 can include a cassette 20, a syringe assembly 300 and a motion transfer cable 200 (e.g., push/pull cable) mechanically coupling the cassette to the syringe assembly. As discussed in greater detail herein, the motion transfer cable 200 can be advantageously actuated (e.g., by a motor or other component of a fluid delivery module) to selectively move a plunger of the syringe assembly 300. Such movement of the plunger relative to the syringe assembly 300 (e.g., into or out of a corresponding syringe barrel or other reservoir) can selectively transfer fluids and/or other materials to or from the syringe assembly. Thus, according to some embodiments, the various injection systems disclosed herein are used to selectively inject fluids into and/or to selectively aspirate fluids from a patient's anatomy (e.g., a joint, an organ, a blood vessel, etc.), as desired or required.

As discussed in greater detail herein, a physician or other clinician can simply grasp the pre-filled syringe and other components of the syringe assembly 300 when performing an injection/aspiration procedure. The force necessary to move the plunger in order to expel fluids and/or other materials from the syringe can be advantageously transferred to the syringe assembly 300 by a motor (or other force-exerting portion or component of the fluid delivery module) via a motion transfer cable 200 (e.g., a push/pull cable or assembly). In other embodiments, the injection system can be used to withdraw fluids from a joint and/or other anatomical location by retracting the motion transfer cable relative to the syringe assembly. Such configurations can be particularly useful when attempting to inject fluids through a relatively small needle (e.g., attached to the distal, output end of the syringe assembly 300) into a region of the anatomy characterized by a relatively high backpressure (e.g., small joints), or to withdraw fluids from such locations. The problem of manually injecting fluids from (or withdrawing fluid from) a syringe can be further exacerbated if the fluids within the syringe are relatively thick, dense or viscous.

The syringe assembly 300, the cassette 20 and/or other components of the injection system can be disposable items that are replaced periodically (e.g., once, twice or more often per day, once a week, once a month, longer or shorter frequencies, etc.) or on an as needed basis. In other embodiments, one or more components of the injection system are configured to be replaced more or less often than indicated herein, as desired or required.

As discussed in greater detail herein, the motion transfer cable 200 (e.g., push/pull cable), and thus the syringe assembly 300, can be actuated by one or more components of the cassette 20. According to some embodiments, the components of the cassette 20 that assist with actuation of the motion transfer cable 200 are operatively coupled to a motor or other movable device of a fluid delivery module. Such motors or other movable devices can operate on a mechanical, pneumatic, hydraulic and/or any other basis.

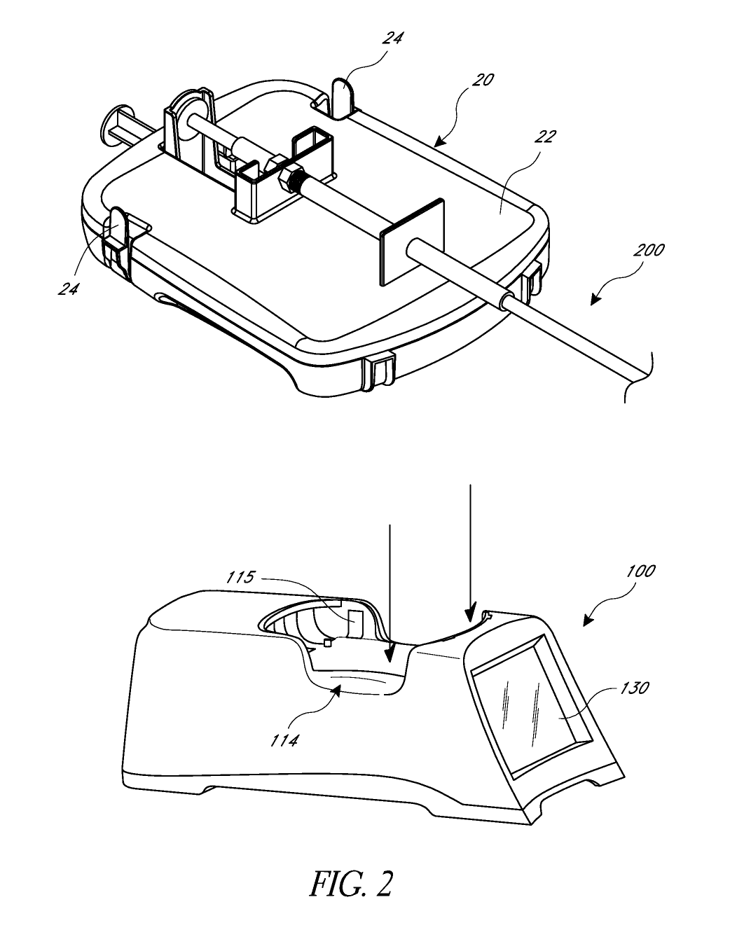

FIG. 2 illustrates one embodiment of a fluid delivery module 100 configured to receive and mechanically couple to one or more movable components of a cassette 20. However, in other embodiments, an injection system comprises a different type of fluid delivery module, motor or other actuation device than those illustrated herein. For example, in some embodiments, the motion transfer cable is directly coupled to a motor or other movable assembly. Such a motor or other movable assembly can be a part of or separate from a fluid delivery module, in accordance with a particular design. In some embodiments, the injection system 10 does not include a cassette 20 and the motion transfer cable 200 is coupled to a stand-alone motor or other movable assembly. Such a stand-alone motor or movable assembly can be placed on a desk in the patient's room, on the bed, attached to the physician or other clinician (e.g., to a cuff or other member secured to a limb or other portion of the clinician's body), etc.

According to some embodiments, a physician or other clinician performing an injection procedure can grasp the syringe assembly 300 and manipulate the assembly 300 so that a needle along its distal end is properly positioned within a patient's anatomy (e.g., a joint, an organ, a blood vessel, etc.). As discussed in greater detail herein, the needle can be located within the anatomy with the assistance of ultrasound or other imaging technologies. Once the syringe assembly has been properly positioned relative to the patient's anatomy, an injection and/or aspiration procedure can be initiated, causing fluids to be delivered out of and/or into the syringe 320 of the syringe assembly 300.

In some embodiments, the motion transfer cable 200 (e.g., push/pull cable) that mechanically couples the syringe assembly 300 to the cassette 20 can comprise the necessary length, flexibility and/or other characteristics to facilitate the handling and manipulation of the syringe assembly 300 by the clinician. Further, as discussed in greater detail below, the injection system can comprise one or more controllers (e.g., button, dials, softkeys, etc.) that allow the clinician to regulate one or more aspects of a delivery/aspiration procedure. Such controllers can be located on a touchscreen 130 of a fluid delivery module, a control module (e.g., configured to be removable coupled to the syringe assembly 300), a remote controller, an integrated ultrasound wand or device and/or the like.

As discussed in greater detail herein, the syringe assembly 300 can be configured to receive a pre-filled syringe and/or any other container comprising one or more fluids, medicaments and/or other materials for delivery into a patient's anatomy. In other embodiments, the syringe assembly 300 is at least partially empty, so as to receive fluids that are aspirated (e.g., from a joint, organ, other portion of the patient's anatomy, etc.) using the injection system. The syringes or other containers configured for placement within the syringe assembly 300 can be of the standard or non-standard type. By way of example, pre-filled syringes 320 or other containers positioned within the syringe assembly 300 of the injection system can include pharmaceutical compositions (e.g., anesthetics, steroids, etc.), hyaluronic acid, drugs, cells, platelet-rich plasma (PRP), other plasma or blood components or formulations, liquid and non-liquid fluids and flowable materials, nanoparticles, cement, microbeads, therapeutics or diagnostic fluids, imaging fluids, lavage fluids, other endogenous or exogenous fluids, etc.

With continued reference to FIG. 2, the cassette 20 can include an outer housing 22 that encloses one or more of its internal components. The cassette 20 can comprise one or more resilient tabs 24, clips, recesses and/or other features that are configured to engage corresponding members, recesses or features 115 on the fluid delivery module 100. For example, in some embodiments, the cassette 20 is secured to the fluid delivery module 100 by urging it within a corresponding recess 114 along the top of the module 100. Tabs 24 on either side of the cassette 20 can engage corresponding recesses 115 or other features of the fluid delivery module. As a result, in some embodiments, the cassette 20 is releasably locked to the fluid delivery module 100.

In one embodiment, in order to release the cassette 20 from the fluid delivery module 100, the tabs 24 are urged toward one another. Accordingly, the interlocking features between the cassette 20 and the module 100 can disengage, permitting the cassette 20 to be lifted or otherwise removed. When lockingly engaged to the fluid delivery module 100, the cassette 20 can be coupled or otherwise movably connected to one or more motors (e.g., stepper motors), actuators and/or other devices or components of the fluid delivery module. In some embodiments, as discussed in greater detail herein, such motors or other movable devices are configured to selectively move (e.g., advance, retract, etc.) one or more corresponding portions of the cassette 20 and motion transfer cable 200 (e.g., push/pull cable) attached to the cassette. As a result, the plunger assembly or other movable device positioned within a pre-filled syringe 320 or other container positioned in the syringe assembly 300 (FIG. 1) can be advantageously moved to transfer fluids and/or other materials to or from a patient. As discussed in greater detail herein, a physician or other clinician can grasp a syringe assembly and selectively manipulate it relative to a patient's anatomy during the execution of a particular injection protocol. The motion transfer cable 200, which in some embodiments mechanically couples the syringe assembly 300 to a fluid delivery module 100 or other movement source can comprise the necessary length (e.g., slack), flexibility and/or other characteristics to facilitate handling and manipulation of the syringe assembly 300 during use.

The incorporation of mechanically (e.g., using a stepper motor), hydraulically, pneumatically or differently driven delivery of medications, formulations and/or other fluids or materials to the patient can facilitate the execution of an injection procedure. For instance, the mechanical transfer of linear movement from the fluid delivery module 100 to the syringe assembly 300 (e.g., via a motion transfer cable 200), as described herein, can permit a physician or other clinician to accurately deliver a volume or other amount of a medicament, formulation or substance (e.g., hyaluronic acid, PRP, steroid, anesthetic, etc.) to a joint. This can be particularly helpful when the manual delivery of such fluids and/or other materials is generally difficult, strenuous, repetitive or otherwise problematic. For example, a relatively high and persistent force and effort may be required by the physician or other clinician to deliver one or more medicaments and/or other substances to a targeted anatomical location. This can be particularly problematic when attempting to inject dense, viscous or high-solids fluids or other materials to small joints (e.g., toes, fingers, midfoot joints, etc.) or other high back-pressure locations within an anatomy (e.g., to or near bones, certain organs, etc.). Thus, at least some of the embodiments of the injection systems, devices and methods disclosed herein permit the delivery of medicaments and/or other materials from a fluid delivery module to a target anatomical location within a patient (and/or to withdraw fluids from a patient's anatomy when the injection system is used in an aspiration mode) without the need to push or exert the necessary force or effort to physically administer such substances. Consequently, the clinician or other user can dedicate more of his or her time and effort in accurately locating a joint or other targeted anatomical location and executing the desired injection procedure.

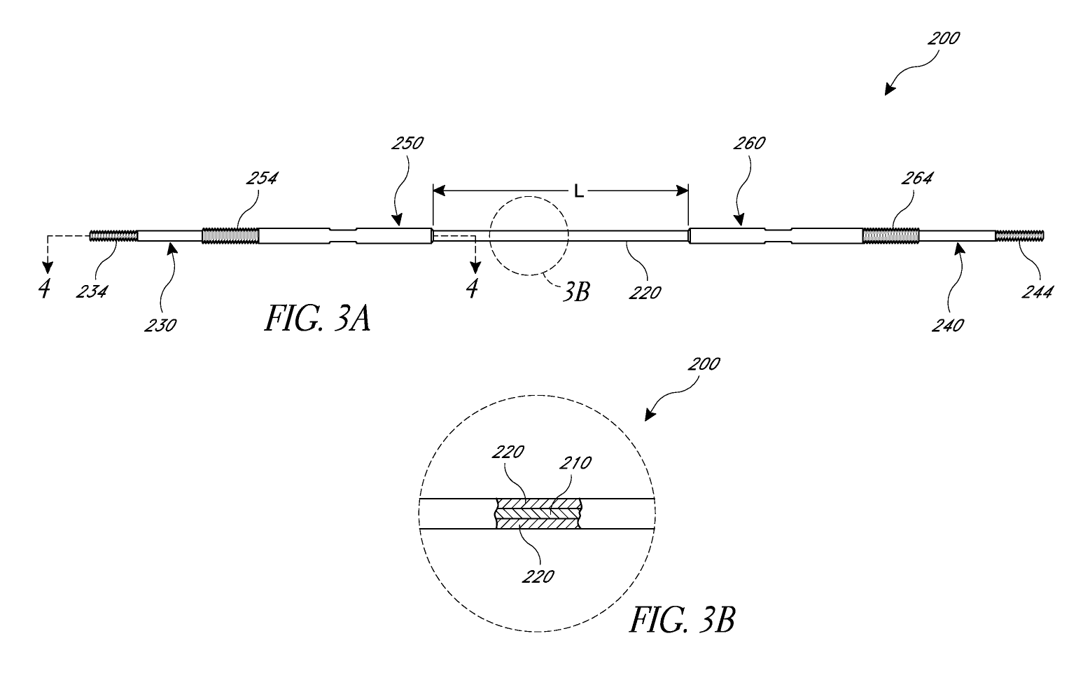

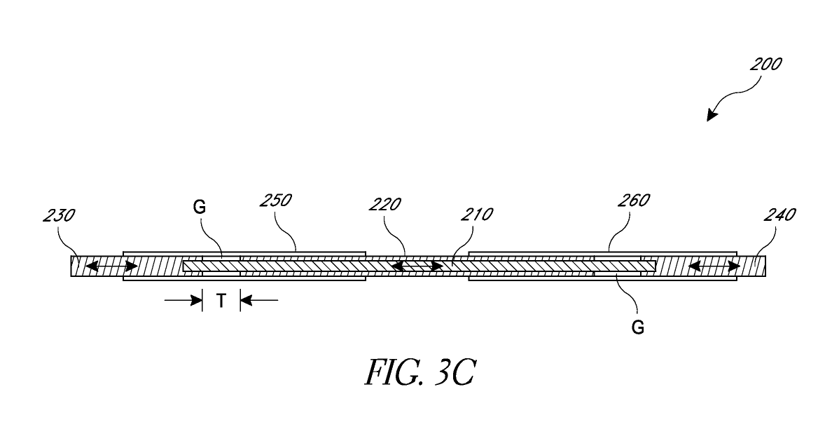

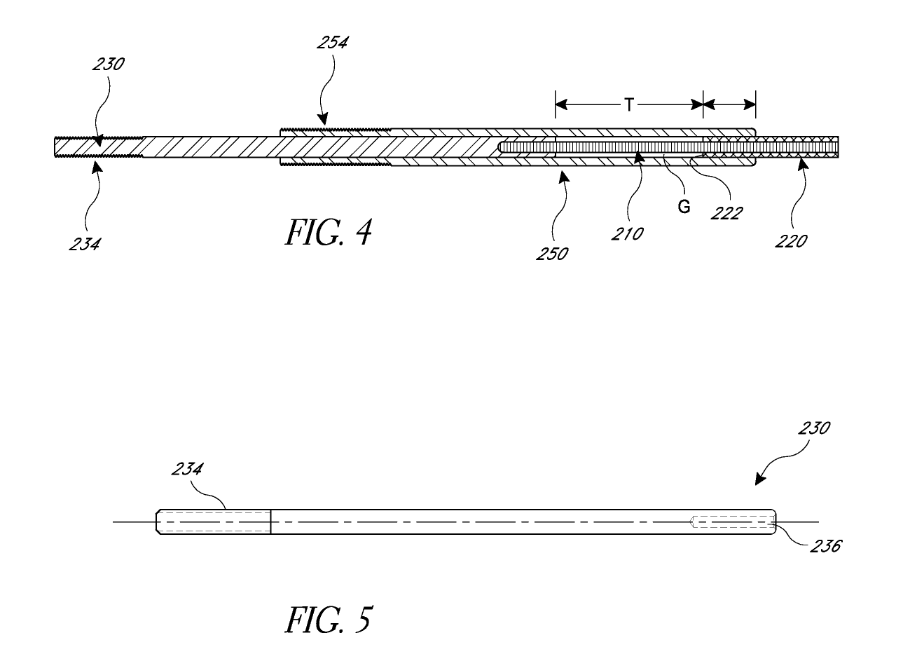

One embodiment of a motion transfer cable 200 (e.g., push/pull cable) for use with an injection system is illustrated in FIGS. 3A and 3B. To more clearly illustrate how the depicted motion transfer cable 200 operates according to some embodiments, a corresponding schematic is also provided in FIG. 3C. As shown, the motion transfer cable 200 can comprise an inner core 210 that is generally surrounded, at least partially along its length, by an outer conduit or sheath 220. In some embodiments, the cable's inner core 210 is slidably moved relative to the outer conduit 220. In several embodiments, the outer conduit or sheath 220 comprises one or more of the following materials: stainless steel, other types of steel, titanium, other metals and/or alloys, plastics or polymeric materials, elastomeric materials and/or the like. In some embodiments, the outer conduit or sheath 220 has a diameter of approximately 0.1-0.4 inches (e.g., about 0.1 inches, 0.15 inches, 0.2 inches, 0.25 inches, 0.3 inches, 0.35 inches, 0.4 inches, values between the foregoing, etc.). In other embodiments, the diameter or other outer dimension of the conduit or sheath 220 is less than about 0.1 inches or greater than about 0.4 inches, as desired or required. In several embodiments, the motion transfer cable 200 comprises one or more of the following materials: stainless steel, other types of steel, titanium, other metals and/or alloys, plastics or polymeric materials, elastomeric materials and/or the like. In some embodiments, the motion transfer cable 200 comprises an outer diameter (e.g., at or along the outer cases 250, 260) of approximately 0.2-0.5 inches (e.g., about 0.2 inches, 0.25 inches, 0.3 inches, 0.35 inches, 0.4 inches, 0.45 inches, 0.5 inches, values between the foregoing, etc.). In other embodiments, the diameter or other outer dimension of the outer cases 250, 260 is less than about 0.2 inches or greater than about 0.5 inches, as desired or required.

With continued reference to FIGS. 3A-3C, the motion transfer cable 200 can comprise a terminal 230, 240 along each of its ends. According to some embodiments, each of the terminals 230, 240 is secured to one end of the inner core 210. Thus, movement of the first terminal 230 along the longitudinal direction of the cable 200 causes the inner core 210 and the second terminal 240 to also move in a similar or identical manner. In some embodiments, the terminals 230, 240 and the inner core 210 are configured to simultaneously move in the same direction and the same distance as each other. Consequently, as discussed in greater detail below, the cable 200 can be advantageously used to transfer linear motion from a motor or other portion of fluid delivery module to a syringe assembly 300 (FIG. 1) that comprises one or more prefilled syringes or other fluid reservoirs. Thus, the mechanical driving component (e.g., fluid delivery module) can be advantageously separated from the syringe assembly 300, thereby simplifying the deign and operation of the syringe assembly 300 and the corresponding procedure in connection with which such an assembly is used.

According to some embodiments, the motion transfer cable 200 additionally comprises an outer case 250, 260 near each of its ends. As shown in FIGS. 3A and 3C, the outer cases 250, 260 can be positioned, at least partially, along the exterior surfaces of the outer conduit or sheath 220 and one of the terminals 230, 240. Each outer case 250, 260 can be immovably coupled to the outer conduit 220 (e.g., using welds, threaded connections, press fit or friction fit connections, adhesives, any other attachment device or method, etc.). As a result, at least in some embodiments, relative movement between the outer cases 250, 260 and the outer conduit 220 is prevented. Similarly, as discussed above, in some embodiments, there is no relative movement between the terminals 230, 240 and the inner core 210. Thus, in such embodiments, the length of the motion transfer cable 200 between the two outer cases 250, 260 remains constant. Further, the terminals 230, 240 and the inner core 210 are permitted to longitudinally move within and relative to the generally unitary structure that is formed by the outer cases 250, 260 and the outer conduit 220.

The inner core 210, the outer conduit 220, the terminals 230, 240, the outer cases 250, 260 and/or any other components or portions of the motion transfer cable 200 (e.g., push/pull cable) can comprise one or more metals, alloys, composites and/or other materials that provide the necessary rigidity, strength, durability, flexibility and/or other desired characteristics to the cable 200. By way of example, the inner core 210, the outer conduit 220, the terminals 230, 240 and the outer cases 250, 260 comprise stainless steel (e.g., surgical steel grade, other grades, etc.) and/or one or more other metals. In some embodiments, the inner core 210 and/or the outer conduit include multiple adjacent sections or are otherwise segmented in order to provide a desired level of flexibility to the motion transfer cable 200. Such a configuration can advantageously provide the clinician with a the necessary slack and flexibility to maneuver the syringe assembly 300 during a procedure.

According to some embodiments, the diameter of the outer conduit 220 and the terminals 230, 240 is approximately 0.2 inches. In other embodiments, however, the diameter of the outer conduit and the terminals 230, 240 can be greater or less than 0.2 inches (e.g., 0.05, 0.1, 0.3, 0.4, 0.5 inches, less than 0.05 inches, greater than 0.5 inches, values between such ranges, etc.), as desired or required. In addition, the length of the motion transfer cable 200 can vary to accommodate a particular injection system design. For example, in one embodiment, the distance between the adjacent ends of the outer cases 250, 260 of the cable 200 (e.g., generally represented by length L in FIG. 3A) is approximately 66 inches. In other embodiments, however, this distance L (or any other length) of the motion transfer cable 200 can be greater or less than 66 inches. In some arrangements, the length of the cable 200 is selected based on the anticipated distance separating the fluid delivery module 100 (FIG. 2) or other actuation device from the patient, the amount of required or desired slack and/or one or more other factors or considerations.

In some embodiments, the general configuration of the motion transfer cable 200 described above, wherein the inner core 210 is adapted to slidably move within the outer conduit or other sheath 220, helps ensure that linear motion of a terminal 230 is accurately and adequately transferred to the opposite terminal 240 while preventing or reducing the likelihood of kinking, bending and/or other problems. At the same time, the cable 200 can be designed with a desired level of flexibility to permit a clinician operating the injection system to maneuver the syringe assembly 300 during the execution of a particular injection/aspiration procedure and/or other protocol.

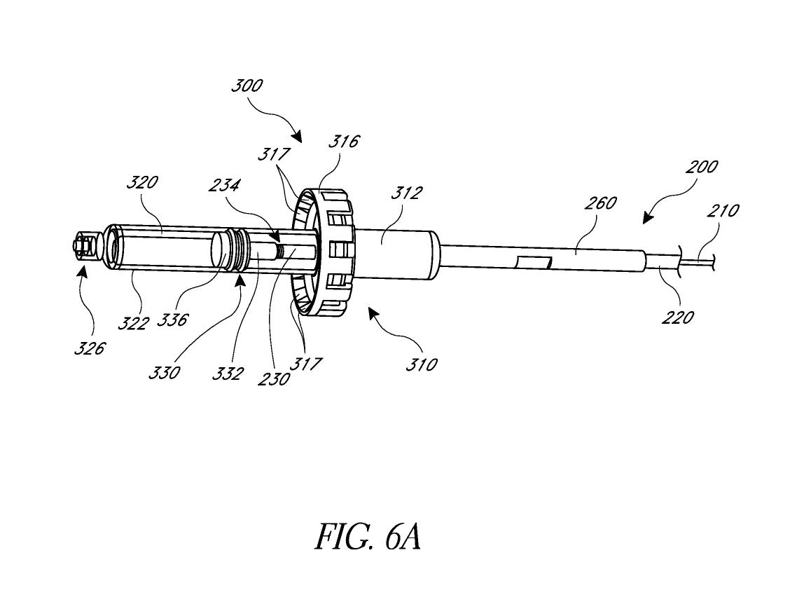

As illustrated in the detailed side view of FIG. 4, a gap G can be provided between the terminal 230 and the outer conduit or sheath 220. This gap G generally represents the distance T that the terminal 230 can be moved (e.g., in the longitudinal direction relative to the outer case 250 and the outer conduit 220) before contacting and generally abutting against the adjacent face or surface 222 of the outer conduit 220. Accordingly, the length T of the gap G can represent the travel of the terminals 230, 240 (and thus the inner core 210) within a particular motion transfer cable 200. In some embodiments, the travel T or a comparable dimension is approximately 2.7 inches and generally corresponds to the distance that a plunger can move (or may be permitted to move) relative to a syringe barrel or other fluid reservoir of the syringe assembly 300. However, in other embodiments, the travel dimension T is greater or less than 2.7 inches (e.g., approximately 1.0, 1.2, 1.4, 1.6, 1.8, 2.0, 2.2, 2.3, 2.4, 2.5, 2.7, 2.8, 2.9, 3.0, 3.2, 3.4, 3.6, 3.8, 4.0, 5.0, 6.0 inches, less than about 1.0 inches, greater than about 6.0 inches, ranges between such values, etc.), as desired or required by a particular syringe assembly and/or other aspect of the injection system.

According to some embodiments, the motion transfer cable 200 can be configured to safely transfer a maximum of approximately 35 pounds of force from one terminal 230 to the opposite terminal 240. However, in other embodiments, the cable 200 can accommodate and transfer more or less than about 35 pounds of force, as desired or required (e.g., about 10, 15, 20, 25, 30, 40, 45, 50, 55, 60, more than about 60, less than about 10 pounds, values between such ranges, etc.). In order to achieve the proper force transfer capacity of a motion transfer cable 200 one or more factors, characteristics or other properties of the cable can be modified, such as, for example, the materials utilized, the diameter, lengths and other dimensions of the cable's components, the types of coupling and/or other connection methods utilized between the various components and/or the like.

With continued reference to FIGS. 4 and 5, one or more of the cable's terminals 230 can include a recess 236 or other opening that is configured to receive an adjacent end of the inner core 210. In some embodiments, the inner core 210 is secured within the recess 236 of the terminal 230 using a threaded or friction fit connection. However, any other type of attachment device or method can be used to couple the inner core to the terminals, such as, for example, adhesives, pins, tabs, welds and/or the like. Further, as illustrated in FIGS. 3A and 4, one or more portions along the ends of the terminals 230, 240 and/or the outer cases 250, 260 can comprise threads 234, 244, 254, 264 and/or coupling features. As discussed in greater detail below, such threaded portions can facilitate attachment of the motion transfer cable 200 (e.g., push/pull cable) to the cassette 20, the syringe assembly 300 and/or any other portion of the injection system. According to some embodiments, the threaded portions 234, 244, 254, 264 of the terminals 230, 240 and/or the outer cases comprise a standard thread design. Other embodiments can include a non-standard thread pattern or any other type of standard or non-standard connection features or portion.

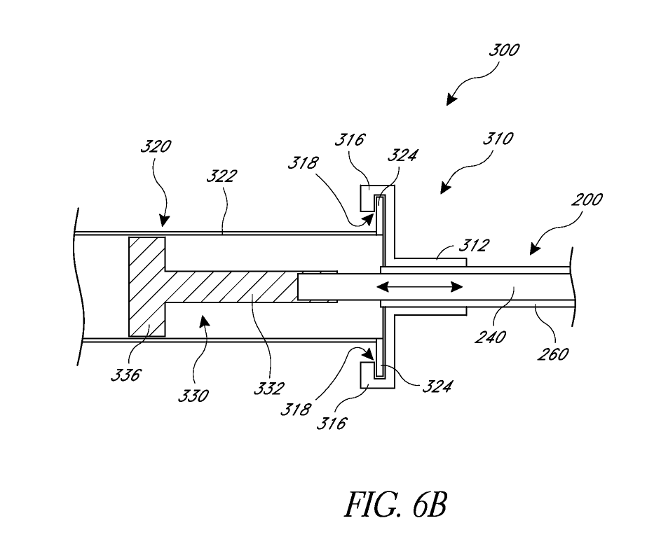

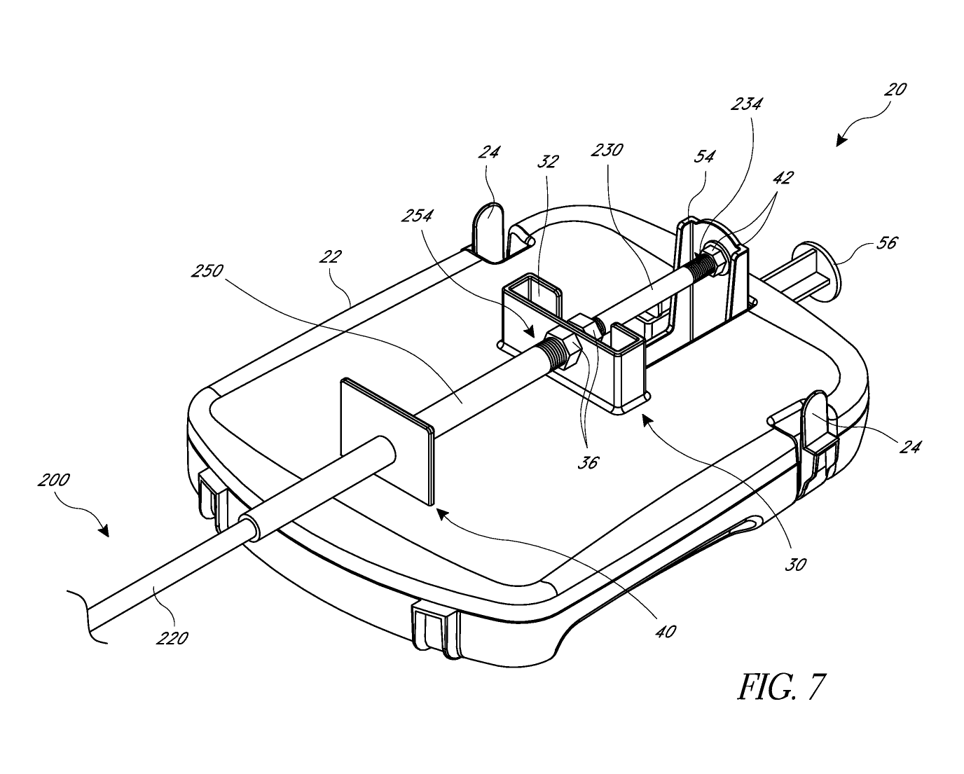

FIGS. 6A and 6B illustrate the connection and interaction details between the syringe assembly 300 and one end of the motion transfer cable 200, according to one embodiment. As noted above, a physician or other clinician can grasp and manipulate the syringe assembly 300 with relation to a patient's anatomy during the execution of a particular injection protocol. The motion transfer cable 200, which mechanically couples the syringe assembly 300 to a fluid delivery module 100 or other movement source can comprise the necessary length (e.g., slack), flexibility and/or other characteristics to facilitate handling and manipulation of the syringe assembly 300.

As shown, the outer case 260 of the cable 200 can be coupled to a syringe adapter 310 of the syringe assembly 300. In one embodiment, the syringe adapter 310 comprises internal threads (or other engagement features) that mate with external threads 264 (or other engagement features) of the outer case 260. The inclusion of threads and/or other engagement features on the outer case 260 and/or the syringe adapter 310 advantageously permits the cable 200 to be easily and securely attached to the syringe assembly 300.

According to some embodiments, the syringe adapter 310 comprises a main shaft portion 312 and a syringe receiving portion 316. As schematically illustrated in FIG. 6B, the syringe receiving portion 316 can be adapted to receive the flange 324 (or other proximal feature or portion) of a syringe 320. In some embodiments, the syringe receiving portion 316 defines a recess 318 or other internal area that can be removably secured to a pre-filled syringe 320. For example, as illustrated in FIG. 6A, the syringe retaining portion 316 of the syringe adapter 310 comprises a plurality of tabs or other engagement portions 317 that are configured to receive and retain a syringe flange. Such tabs 317 can be resilient to permit a syringe flange 324 to be selectively secured to or removed from the syringe retaining portion 316. In other embodiments, one or more other features, devices and/or methods can be used to secure a syringe 320 to the syringe receiving portion 316 of the syringe assembly 300.

With continued reference to FIGS. 6A and 6B, one of the terminals 240 of the motion transfer cable 200 can be removably attached to a plunger 330 of the syringe assembly 300. In some embodiments, at least a portion of the terminal 240 comprises external threads (and/or other engagement features) that are configured to mate with corresponding threads (and/or other engagement features) of the plunger's shaft portion 332. In some embodiments, the thread pattern of the terminal 240 is standard and configured to mate with a standard corresponding thread pattern of the plunger 330. Regardless of how the terminal 240 is coupled to the plunger 330, the cable 200 can be configured to accurately slidably move the plunger 330 into and/or out of the syringe 320 in order to transfer fluids to or from a patient's anatomy. As the terminal 230 located on the opposite end (not shown) of the motion transfer cable 200 is advanced or retracted (e.g., by a motor or other portion of the fluid delivery module), the inner core 210 and the terminal 240 that is coupled to the syringe assembly 300 will move accordingly. For example, in order to deliver a desired volume of fluid and/or other material stored within the syringe 320, the fluid delivery module can move the opposite terminal a specific distance in the forward direction (e.g., toward the outer conduit 220 and the syringe assembly 300). Accordingly, the terminal 240 will urge the plunger assembly 330 into the reservoir of the syringe 302. As a result, the desired volume of fluids and/or other materials pass from the interior of the syringe barrel 322 through the outlet 326 along the distal end of the syringe 320.

According to some embodiments, the outlet 326 of the syringe 320 or other container that is coupled to the cable 200 can include a luer lock fitting and/or any other standard or non-standard connection. For example, the outlet 326 can include a threaded connection, a flanged connection, a snap-fit, pressure-fit or friction-fit connection and/or the like. Thus, a needle assembly (not shown) having a corresponding connection feature or device can be easily and quickly connected to and/or disconnected from the outlet 326 of the pre-filled or other type of syringe 320.

In some embodiments, the needle assembly secured to the distal end of the syringe assembly 300 includes a needle that can be advanced through the skin and other tissues of a patient so as to adequately reach a targeted joint (e.g., toe, ankle, knee, spine, hand, finger, neck, etc.) or other anatomical location (e.g., organ, cyst, cavity, etc.). In several embodiments, the needle has a gauge of 18 G-30 G and a length of about 0.5 to 5.0 inches (e.g., 1.0 to 1.5 inches). However, in other arrangements, the gauge, length and/or other details of the needle can be greater or smaller than the range indicated herein, as desired or required by a particular application. Further, the needle can comprise surgical-grade stainless steel and/or any other suitable material (e.g., other metals, alloys, etc.).

Once a desired volume of the fluid and/or other materials contained within a syringe 320 has been delivered to a patient, the disposable needle attached to the distal outlet 326 of the syringe 320 can be discarded. In other embodiments, the syringe is decoupled from the syringe receiving portion 316 of the syringe assembly 300 and discarded together with the needle, depending on the specific injection/aspiration protocol desired or used. In other embodiments, as discussed herein, the syringe assembly can be used to retain aspirated fluids withdrawn from a patient's anatomy (e.g., joint, organ, etc.). In such arrangements, the motion transfer cable is withdrawn or retracted relative to the syringe so as to create a vacuum or negative pressure within the syringe interior and corresponding needle.

As noted above, the syringe assembly 300 can be advantageously configured to accommodate syringes (or other fluid containers) of various sizes, shapes and/or other characteristics. For instance, the syringe receiving portion 316 can be sized, shaped and otherwise adapted to securely receive standard or non-standard syringes having varying types (e.g., standard or non-standard) of proximal flanges 324. Further, the terminal 240 can be sized, shaped and otherwise configured to mate with a proximal end of the plunger assembly 330 (e.g., via a standard or non-standard threaded connection). In some embodiments, plunger assemblies 330 are provided that are specifically intended to couple to the adjacent terminal 240. Thus, in such embodiments, plunger assemblies 330 can be provided in a variety of lengths, diameters, stopper size, other dimensions and/or other characteristics.

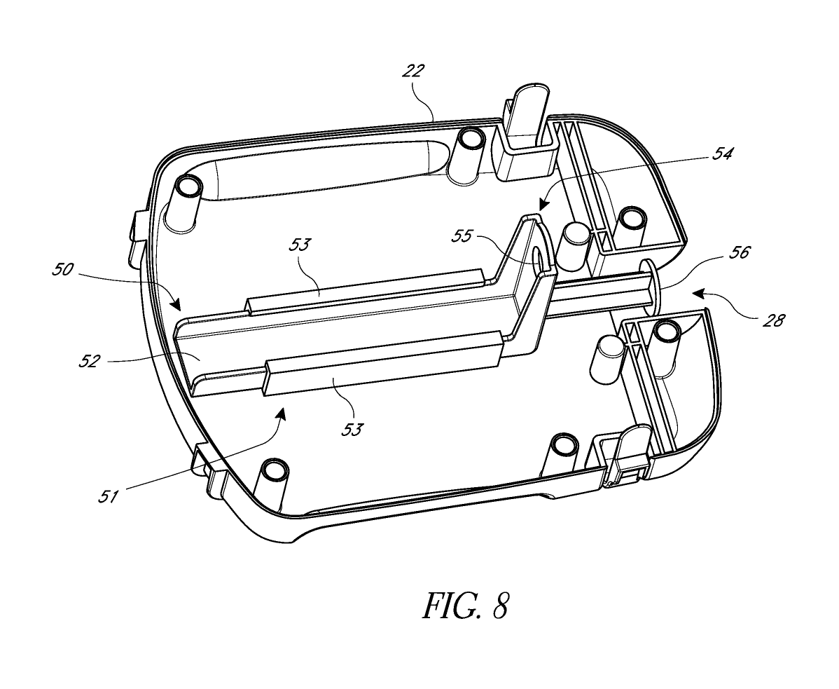

FIG. 7 illustrates one embodiment of a cassette 20 sized, shaped and otherwise configured to be secured to a fluid delivery module 100 (FIG. 2) or other actuation device capable of linearly advancing a terminal 230 of the motion transfer cable 200. As shown, an outer housing 22 can comprise one or more anchors 30, 40, retention features and/or other devices or features that help secure the proximal end of the cable 200 to the cassette 20. For example, in the depicted embodiment, the cassette 20 comprises a first retention assembly 30 having at least one vertical wall 32 that extends upwardly from the housing 22. In some arrangements, the wall 32 includes an opening through which a portion of the motion transfer cable 200 may be passed. As shown, for instance, the retention assembly 30 can be sized, shaped and otherwise adapted to receive the outer case or sheath 250 portion of the cable 200. According to some embodiments, threaded nuts 36 can be used to secure the outer case or sheath 250 to the cassette. For example, as illustrated in FIG. 7, nuts 36 can be positioned along threaded portion 254 of the outer case 250 on either side of the retention assembly's wall 32. Any other securement device or feature can be used to secure a portion of the cable 200 to the cassette 20, either in addition to or in lieu of nuts.

With continued reference to FIG. 7, the cassette 20 can comprise one or more other retention assemblies or guides 40 to properly secure and/or align the cable 200 to the cassette, to help the cassette accommodate any forces, moments and/or other stresses to which it may be subject during use (e.g., when the terminal 230 is linearly moved by the fluid delivery module) and/or for any other purpose. For instance, as illustrated in FIG. 7, the cassette 20 can include a second guide 40 that extends upwardly from the housing 22 and has an opening (e.g., to also receive a portion of the cable's outer case 250). In other embodiments, the quantity, type, size, shape and/or other details of any cable retention features, guides and/or the like can be different than disclosed herein, as desired or required.

In FIG. 7, the terminal 230 of the motion transfer cable 200 can also be coupled to the cassette 20 and slidably moved using a riser 54 of the cassette's actuator member. As shown, the riser 54 can extend from an interior of the cassette through a slot or other opening along the top surface housing 22. In some embodiments, the riser 54 is secured to the terminal 230 with the help of nuts 42 or other securement devices positioned on either side of the riser 54 (e.g., along or near the threaded portion 234 of the terminal 230). However, in other embodiments, one or more other devices, features or methods can be used to couple and secure the terminal 230 to the riser 54 and/or one or more other portions of the cassette's actuator interface.

Once the terminal 230 and the outer case 250 have been securely coupled to corresponding portions of the cassette (e.g., as illustrated in FIG. 7), the terminals 230, 240 and the inner core 210 can be moved in unison by a corresponding movement of the riser 54. For example, when the riser 54 is moved distally (e.g., toward the outer case 250), the terminal 230 urges the inner core 210 and the opposite terminal 240 in the same direction. Consequently, as discussed above, the plunger assembly 330 is urged within an interior of the syringe 320, causing fluids and/or other materials contained therein to be transferred through the syringe outlet 326 and a distal needle assembly. Thus, the manner in which the riser 54 is moved will determine how a particular injection procedure is executed. For example, the total volume of fluids discharged from the syringe 320 will depend on how far the riser moved. Likewise, the flow rate of injected fluids will depend on the rate at which the riser is moved. Accordingly, in some embodiments, the fluid delivery module of the injection system is configured to accurately linearly advance the riser to ensure that fluids, medicaments and/or other materials contained within a particular syringe 320 are safely and accurately administered within a patient.

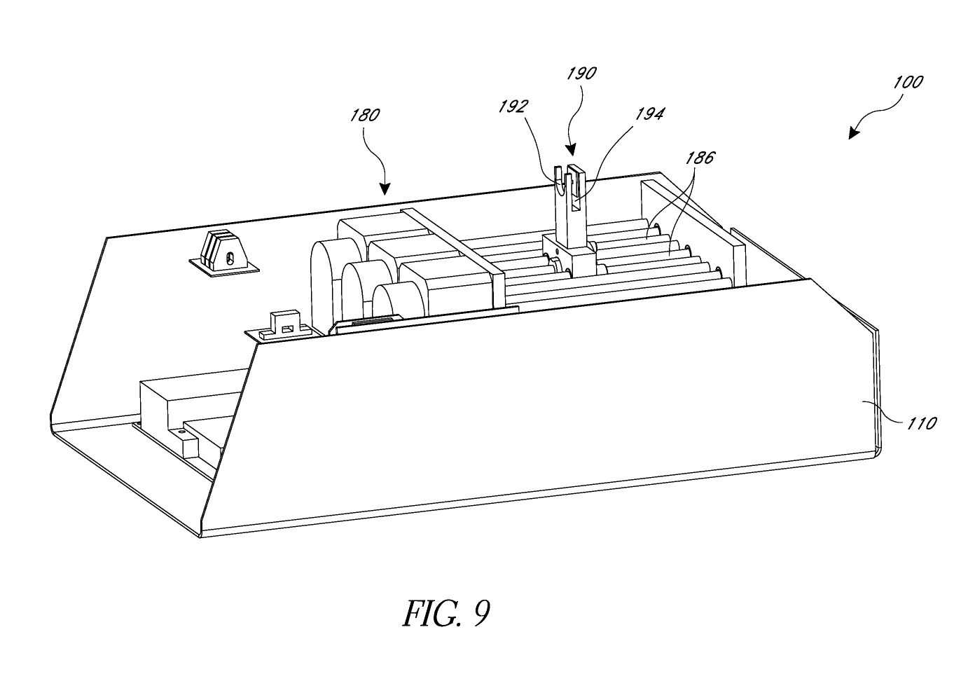

FIG. 8 illustrates an interior view of the cassette 20 comprising a riser 54 that extends above the housing 22 and is coupled to the cable's terminal 230. As shown, the riser 54 can be part of an actuator member 50 that includes a main portion 52 that is adapted to slidably move within a rail system 51 located along or near the bottom surface of the cassette interior. In some embodiments, the rail system 51 comprises two channels 53 that are shaped, sized and otherwise configured to create a space through which the main portion 52 of the actuator member 50 can slide.

With continued reference to FIG. 8, the actuator member 50 comprises a riser 54 that extends generally vertically from the main portion 52. In some embodiments, as discussed above, the riser 54 includes one or more openings 55 or other features (e.g., slots, recesses, flanges, etc.) that are sized, shaped and otherwise configured to receive the threaded portion 234 of the terminal 230 and help secure a movable portion of the motion transfer cable 200 (e.g., push/pull cable) to the cassette 20. Accordingly, movement of the actuator member 50 relative to the cassette housing 22 (e.g., within the rail system 51) can cause the terminals 230, 240 and the inner core 210 to linearly move relative to other components of the cable 200, resulting in a corresponding movement of the plunger assembly that is coupled to the other end of the cable 200.

As illustrated in FIG. 8, the actuator member 50 can include a stem 56 or other member that extends from the main portion 52 and the riser 54, toward the rear of the cassette 20. In some embodiments, the stem 56 is generally aligned with a slot or opening 28 along the bottom surface of the cassette housing 22. Accordingly, the stem 56 can be secured within a pusher block or other portion of a fluid delivery module (FIG. 2). As a result, a motor (e.g., stepper motor), other mechanically-operated movement system and/or any other device or system of the fluid delivery module (or other device into which the cassette 20 is inserted or to which the cable is coupled), can advantageously manipulate the stem 56 to selectively move the terminals 230, 240, the inner core, and thus, the plunger assembly 330 relative to its barrel portion 322 of the syringe 320. As noted above, urging the plunger member within the interior of the barrel portion helps to transfer fluids and/or other materials contained out of the pre-filled syringe 320 or other container positioned within the syringe assembly 300.

According to some embodiments, a fluid delivery module 100 of the injection system includes one or more motors, pumps, other fluid transfer devices (e.g., devices or components operated by a motor, actuator and/or other mechanical device) to help transfer one or more medicaments, fluids and/or other substances or materials from the syringe 320 to a targeted anatomical location (e.g., toe, knee, other joint, etc.). In some embodiments, the motor comprises a stepper motor or another type of motor that is configured to accurately move the motion transfer cable (e.g., in a generally linear manner) in order to transfer a volume of fluid and/or other material from the syringe to or near a joint or another targeted anatomical location of a patient. Stepper motors or other mechanically-driven motors or devices can be especially helpful in executing such intra-articular injections (e.g., due to, in part, the relatively high backpressure associated with injecting fluids and/or other materials into small joint, the innervated nature of such joints, the need to deliver very accurate volumes of fluids and/or other materials to such joints and/or other factors or reasons).

In some embodiments, the fluid delivery module 100 is an electromechanical software-controlled device that uses one or more motors (e.g., stepper motors), pumps and/or other mechanical, pneumatic, electrical or other types of devices to selectively expel fluids and/or other materials from a pre-filled syringe or other container accommodated within the syringe assembly. In one embodiment, the system comprises one or more stepper motors and/or other mechanically-operated actuators or devices to accomplish the accurate delivery of fluids. Further, in some embodiments, a needle positioned at the distal end of the syringe assembly 300 is used to aspirate fluids from a targeted anatomical location (e.g., a joint, an organ, a cyst, another body cavity, etc.). As discussed in greater detail herein, such aspiration or fluid removal procedures can be performed by retracting the plunger assembly from the syringe barrel.

In other embodiments, the fluid delivery module is a simply device comprising a motor or other mechanical assembly. In some embodiments, such a simplified fluid delivery module does not comprise a touchscreen display, a user input devices, a display and/or the like. Therefore, in its simplest form, the fluid delivery module can comprise only one or more motors or other devices configured to selectively move the motion transfer cable (e.g., either toward or away from the syringe assembly).

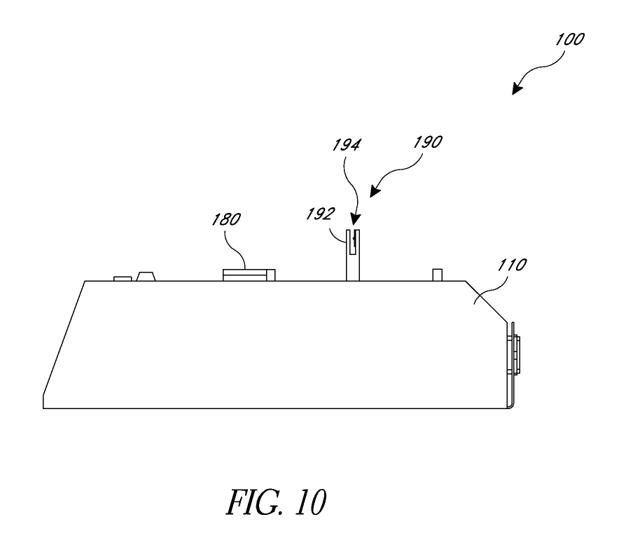

One embodiment of a fluid delivery module 100 configured to accurately move the riser 54, and thus the motion transfer cable 200 and the plunger assembly 330 coupled thereto, is illustrated in FIGS. 9 and 10. As shown, an interior of the fluid delivery module 100 can comprise one or more stepper motors 180 or other devices (e.g., other motors, other mechanically-actuated devices, pneumatically or hydraulically actuated devices, etc.) configured to selectively move the actuator member 50 of the cassette and the movable portions of the motion transfer cable 200 and the syringe assembly 300 to which the actuator member 50 is coupled. As discussed in greater detail herein, such linear movement of a motor 180 can facilitate the accurate transfer of fluids and/or other to or from a syringe or other container positioned within the syringe assembly 300 of the injection system.

With continued reference to FIGS. 9 and 10, the stepper motor 180 can be adapted to selectively move a corresponding pusher block 190 along one or more guide rails 186. In the depicted embodiment, the pusher block 190 is configured to move linearly relative to two guide rails 186. However, in other embodiments, a pusher block 190 is configured to move in two or more directions, along more or fewer guide rails and/or in a completely different manner, as desired or required.

In the illustrated embodiment, the pusher block 190 includes a vertical portion 192 that is sized, shaped and otherwise adapted to engage the stem 56 of the cassette's actuator member (FIG. 8). As discussed herein with reference to, inter alia, FIG. 8, a cassette 20 can include one or more openings 28 adjacent to the stem 56 (e.g., along the bottom of the cassette housing, along any other surface or portion of the cassette, etc.). Accordingly, the vertical portion 192 of the pusher block 190 can be configured to extend through such an opening 28 of the cassette 20 in order to engage the stem 56.

In FIGS. 9 and 10, the vertical portion 192 of the pusher block 190 comprises a slot 194 that is sized, shaped, positioned and otherwise configured to securely receive a corresponding portion of the stem or another component of the cassette's actuator member 50. Thus, as the pusher block 190 is moved along the guide rails 186, the position of the stem 56 and the riser 54 can be selectively adjusted. As discussed in greater detail herein, this permits the terminals and inner core of the motion transfer cable 200 to move the plunger assembly 330 into the syringe reservoir, as desired or required. Consequently, a volume of fluids and/or other materials stored within the syringe 320 can be accurately delivered to a targeted anatomical location (e.g., a joint, an organ, a cavity, etc.). Thus, such fluids and/or other materials can be transferred from a syringe (e.g., pre-filled syringe) or other container with reliable precision and with relative ease. In order to ensure that the position of the pusher block 190 is being accurately controlled, the fluid delivery module 100 can comprise one or more sensors (e.g., optical sensors), other position detection devices and/or the like.

As discussed herein, for any of the injection systems disclosed herein, the stepper motor or other device of the fluid delivery module can be configured to move the motion transfer cable toward the syringe assembly (e.g., to selectively delivery fluids and/or other materials into a patient's anatomy) and/or away from the syringe assembly (e.g., to selectively aspirate or withdraw fluids and/or other materials form a patient's anatomy). Additionally, for any of the injection systems disclosed herein, the cassette 20 may be optional, and the motion transfer cable 200 can be coupled to a stand-alone motor or other device capable of moving the motion transfer cable 200.

According to some embodiments, a control module can be removably attached to an exterior portion of the syringe assembly 300. Such a control module can include one or more clips, tabs and/or other securement members that are adapted to facilitate attachment to and detachment from the syringe assembly 300. For example, a securement member can include a protruding portion or feature that is sized, shaped and otherwise configured to mate with a corresponding opening, recess or other feature of the syringe adapter and/or other portion of the syringe assembly.

According to some embodiments, the control module comprises one or more buttons controllers and/or other adjustment devices (e.g., knobs, dials, switches, etc.). Such buttons or other controllers can help regulate the delivery of the fluid and/or other material contained within the syringe 320 of the syringe assembly 300. For example, buttons can be used to activate or deactivate (e.g., ON/OFF) the delivery of such a fluid, medicament or other substance to a patient's anatomical location (e.g., joint, organ, cavity, etc.). In certain embodiments, the buttons or other controllers are manipulated to regulate the rate of delivery (e.g., flowrate) of the medicament and/or other material being discharged from the syringe 320. As discussed in greater detail herein, in embodiments where the fluid delivery module is in data communication with one or more other components or devices (e.g., ultrasound devices, radio frequency spectroscopy devices, other imaging devices or systems, etc.), one or more buttons or other controllers on such a removable control module and/or other portions of the injection system can be used to help regulate the operation of such systems. For example, one or more buttons or other controllers of a control module can be used to capture an ultrasound image or video while a target anatomical space (e.g., joint, organ, etc.) is being located and/or while fluids or other materials are being injected into a target anatomical location. Alternatively, the buttons or other controllers can be used to vary one or more other aspects of an imaging system, such as, for example, zoom, resolution, contrast, brightness and/or the like. In some embodiments, a control module includes additional, fewer and/or different buttons, knobs, levers and/or other control devices that permit a user to control one or more aspects of the injection system. In other arrangements, one or more operational aspects of the injection system (e.g., the distance by which the motion transfer cable 200 is moved) is controlled using other types of controllers, such as, for example, the touchscreen 130 of the fluid delivery module, a foot pedal, an audible instruction receiving devices and/or the like, as desired or required.

Regardless of their exact quantity, type, configuration and/or other details, buttons or other controllers can be used to initiate and/or terminate a particular injection procedure. Thus, once a particular injection protocol has been selected by a user (e.g., through the display or other interface of the fluid delivery module), pressing a button and/or otherwise manipulating another controller can regulate one or more aspects of the delivery of fluids and/or other materials through the syringe assembly 300. For example, by pressing or manipulating a button once, the injection procedure can be initiated. In some embodiments, additional manipulation of such a button or other controller can terminate or temporarily pause the procedure. In yet other embodiments, manipulation of the button or other controller can alter the flowrate, sequence and/or any other aspect of an injection procedure.

According to certain embodiments, a control module and/or any other portion of an injection system comprises one or more two-mode or other multi-mode buttons and/or other controllers. Pressing or otherwise manipulating such a button can commence or terminate (e.g., in an alternating pattern) the delivery of fluids and/or other materials from the syringe assembly 300. Alternatively, an injection system can include one or more other types of buttons or controllers. In some arrangements, such buttons or other controllers are configured to permit the user to select between two, three or more different settings. In other embodiments, a button is of the multi-depth type (e.g., dual-depth, tri-depth, etc.), enabling a user to selectively press the button to two or more distinct depths or other levels. Each distinct depth or level can correspond to a particular setting (e.g., flowrate, selection of which fluids or other materials to deliver, etc.). For example, pressing the button to the first level can cause the desired fluid and/or other material to be conveyed at the maximum or minimum rate. Further, continuing to press the button to subsequent lower levels can cause the rate of delivery to increase, decrease or terminate. In other embodiments, the controller comprises multi-depth buttons that do not include distinct depths, such as, for example, a rheostat. Thus, a particular setting (e.g., flowrate) can be varied based on the depth to which a button is depressed.

In other arrangements, a control module of the injection system comprises one or more buttons that have only two positions, but which are configured to permit a user to select between three or more different settings. For example, an injection system can be adapted to sequentially move between different flowrate settings (e.g., high-medium-low-off, vice versa, etc.) every time such a button is pressed.

As discussed herein, a control module and/or another portion of an injection system can comprise other types of controllers, either in lieu of or in addition to buttons. For example, an injection system can include a roller ball, a roller wheel, a dial, a knob, a modulating switch or other device and/or the like. Regardless of their exact configuration and design, such control devices can enable a clinician or other user to regulate the delivery of fluids and/or other materials from the fluid delivery module to a patient. According to some embodiments, a user can pre-select a desired injection protocol, which includes the rate of delivery, the volume or other amount to be delivered and/or other details for the medicament, other fluid and/or other substance used in a particular treatment. Thus, a user simply has to press, release or otherwise manipulate the button or other controller of the control module in order to initiate, terminate, pause or otherwise alter the preselected protocol.

In other embodiments, one or more buttons of the control module are adapted to guide the user through one or more user-interface screens on the display or graphic user interface (GUI) on the fluid delivery module. Thus, the buttons and/or other controllers located on a control module can be used to make selections through one or more menus or the like.

In any of the embodiments disclosed herein, a control module can be connected to the fluid delivery module of the injection system using a radio frequency (RF) or other wireless connection (e.g., Bluetooth, Wi-Fi, etc.). However, the control module can be configured to communicate with the fluid delivery module and/or any other component of the injection system using a hardwired connection, either in addition to or in lieu of a wireless connection. A control module can comprise one or more disposable or rechargeable batteries (e.g., standard or non-standard batteries, battery packs, etc.). In some embodiments, the batteries of the control module are configured to be recharged when the control module is placed within or near a docking station or other recharging location (e.g., of the fluid delivery module, other portion or component of the injection system, etc.). In some embodiments, batteries (e.g., standard or non-standard batteries, battery packs, etc.) configured to power the control module can be removed from the control module before being recharged (e.g., by placement in or near a recharging device or location). In some embodiments, the fluid delivery module or another component of the injection system comprises a docking station that is adapted to recharge a battery using electromagnetic induction, simple charging (e.g., using a DC or AC connection), pulse charging and/or any other charging method or device. Thus, in some arrangements, the batteries within the control module can be permitted to recharge when the syringe assembly is not in use. Alternatively, the control module can be configured to draw its power from one or more other sources, such as, for example, a DC or AC hardwired connection and/or the like. In yet other embodiments, an injection system comprises two or more control modules, allowing one or more modules to be recharged while a syringe assembly 300 is being utilized.

In addition to batteries, an interior portion of the control module can include circuitry, indicator lights (e.g., LEDs) and/or any other component or feature. For example, the control module can include one or more indicator lights that provide information to the clinician or other user of the assembly prior to, during and/or following an injection procedure. For example, an LED or other indicator light can be configured to light up when the battery power of the module is above or below a particular threshold level (e.g., adequately charged, in need of charging, etc.). Alternatively, the brightness, color and/or other characteristics of the indicator light can be configured to change in response to certain conditions. For instance, the properties of the light can vary based on the strength of the battery, on the signal strength of the wireless connection (e.g., RF, Bluetooth, etc.) between the control module and the fluid delivery module and/or another component of the injection system and/or any other aspect associated with the injection system.

In some embodiments, other data or information about the fluids and/or other materials that are loaded into the syringe assembly 300 can be provided on the screenshots. For example, information about the name of the composition and/or other fluid or material can be provided. In other arrangements, a code (e.g., NDC) and/or other identifier about the particular medication or formulation secured within the syringe assembly 300 can be displayed. Further, as discussed in greater detail herein, the pre-filled syringes and/or other containers secured to the syringe assembly 300 can be configured to be automatically or manually identified (e.g., using an identification flag or other member, using a barcode scanner or other identification device positioned along the outside of the fluid delivery module, etc.). Thus, information detected by such devices (e.g., type of medication, dosage or concentration, manufacturer, expiration date, etc.) can be advantageously provided on the display of the fluid delivery module. In addition, other data or other information can also be included on the display, such as, for example, imaging data for locating the distal end of the needle, date, time, name of the patient, name of the physician or other clinician performing the procedure and/or the like, as desired or required.

In some embodiments, the touchscreen display includes up and down arrows associated with the medication, formulation and/or other fluid or material to be delivered to a patient in a scheduled injection procedure. Thus, a clinician or other user can select the volume, mass and/or other amount of a particular substance that should be delivered within a targeted anatomical location for an injection procedure. The volume or other amount selected at any particular time can be displayed in a corresponding area of the display. In addition, the total volume or other measure of fluids and/or other materials to be delivered within an anatomy for a particular injection procedure can also be displayed.

According to some embodiments, the touchscreen display offers a convenient way of modifying a particular protocol using the up and down arrows. In addition, the touchscreen display can include one or more softkeys or other buttons (e.g., “FLUID SET-UP”, “SYSTEM SET-UP”, “CASSETTE REMOVAL”, “DATE TIME”, “MENU”, “OK”, etc.) that enable a user to input desired settings (e.g., maneuver through the various screens) and/or adjust the details associated with a specific injection procedure.

In some embodiments, once the details of a desired injection protocol have been entered, a clinician or other user can use a foot pedal or other separate controller (e.g., a controller that is selectively attachable to the syringe assembly) to regulate the delivery of fluids and/or other materials to a patient. According to some embodiments, the rate of delivery of a medication, formulation and/or material within the syringe is adjusted.

According to some embodiments, a fluid delivery module used in connection with one or more of the injection system configuration disclosed herein can include one or more displays 130 or other user interfaces along one or more of its outer surfaces (FIG. 2). As discussed in greater detail herein, the display 130 can be configured to provide various data and/or other information to the user. In some embodiments, the fluid delivery module 100 comprises a data input device (e.g., touchscreen, keyboard, keypad, dials, buttons, etc.) to permit a user to enter data and/or other information regarding a particular procedure. For example, in one arrangement, the display 130 comprises a touchscreen configured to both provide information to and receive information and instructions from a user.

In some embodiments, as illustrated in FIG. 2, a touchscreen display 130 of a fluid delivery module 100 is generally rectangular. In certain arrangements, the display 130 comprises a flat panel touchscreen having a 7-inch color TFT LCD. The resolution of the display 130 can be approximately 800×600 with a total of about 480,000 pixels and a brightness rating of approximately 300 cd/m³. In addition, the touchscreen display 130 can use restive technology for sending touch input. In some embodiments, the touchscreen is compatible with and/or without the use of gloves (e.g., latex gloves). However, the type, size, resolution, brightness, compatibility and/or other details about the display 130 can vary, as desired or required.

In some embodiments, the touchscreen display 130 can comprise a 16 to 9 aspect ratio. However, as noted above, the type, shape, size, aspect ratio, resolution and/or other characteristics of the display 130 can vary, as desired or required. As discussed in greater detail herein, the touchscreen display 130 can be adapted to identify one or more characteristics regarding the syringes or other containers positioned within the syringe assembly 300. In addition, the touchscreen display 130 can be configured to display status information, patient information (e.g., name, vital signs, known allergies, etc.), imaging information, injection procedure programming and/or status information and/or any other information. Further, the touchscreen display 130 and/or another data entry device can permit a physician, other clinician or other user to control the operation of the procedure (e.g., verify patient, verify fluids or other materials to be delivered, locate target joint, start, stop, reduce/increase flowrate or other rate of delivery, etc.) and/or to enter other data within the system 10.

According to some arrangements, the touchscreen display 130 is configured to illustrate text and/or images (e.g., icons). The use of icons can facilitate the physician or other user in performing the required injection and/or aspiration procedure. For example, the touchscreen display 130 can be configured to display a list of various body parts (e.g., foot, hand, spine, knee, other body parts or organs, etc.) into which a desired injection and/or aspiration procedure is to occur. Once a user selects the general anatomical area targeted by the procedure, the touchscreen display 130 can provide a more detailed selection list of available target sites within that general area. For example, if a foot is selected, the touchscreen display 130 can provide a more detailed list of joints associated with the foot (e.g., ankle, toe, etc.). Further, the display 130 can provide a list of various treatment procedures or injection protocols from which to choose. In other embodiments, the touchscreen display 130 can include “UP” and “DOWN” softkeys, arrows, other icons, text and/or other images that facilitate the user during the execution of the corresponding procedure. For instance, such softkeys can allow a clinician to select a volume of the pre-filled syringe that will be transferred to the patient, the rate of delivery and/or the like.

In some embodiments, the selected icon or other portion of the display 130 can be configured to change color, shade, shape and/or the like when a user selects it. Further, the fluid delivery module 100 can be configured to provide visual and/or audible verification that a selection was made (e.g., tone, beep, etc.). In some embodiments, a touchscreen display 130 and/or any other component of the fluid delivery module 100 includes one or more other features, as required or desired by a particular application. As discussed, an injection system can also include a voice command/notification system that permits a user to receive audile updates from the system (e.g., volume dispensed, volume remaining, etc.) and/or to control the operation of the system using audible instructions (e.g., “START,” “STOP,” “DECREASE DELIVERY RATE,” “INCREASE DELIVERY RATE,” “PAUSE,” “TERMINATE” and/or the like). The disclosure included herein regarding the display 130 (e.g., touchscreen device) and other features of the injection system can be applied to any other embodiment of a fluid delivery module disclosed herein or equivalents thereof.

The fluid delivery module 100 and/or any other components of the injection system 10 can be electrically energized by one or more power sources. For example, in some embodiments, the fluid delivery module 100 is configured to connect to an AC power supply (e.g., via a cord or other connection). In such arrangements, an AC transformer can be situated either within or outside of the module housing 110. Thus, in some embodiments, a fluid delivery module includes an external power supply. In other embodiments, however, the fluid delivery module is powered by one or more batteries (e.g., rechargeable lithium batteries, disposable batteries, etc.) or another DC power source, either in addition to or in lieu of an AC power supply. This can provide an extra measure of protection to ensure that an injection procedure is not interrupted because of a power outage or other disruption. In addition, the use of batteries and/or an external AC power transformer can advantageously enhance the portability of the injection system and/or help to reduce its overall size and/or weight. However, in alternative embodiments, one or more other types of devices and/or methods are used to provide electrical power to the fluid delivery module 100 and/or other components of the injection system 10.

According to some embodiments, a fluid delivery module 100 includes one or more other ports, slots and/or other connection sites configured to operatively connect the module 100 to one or more other devices, processors and/or the like (e.g., ultrasound or other imaging device, personal computer, internet, other local or non-local network, etc.). Such ports or slots can be standard (e.g., USB, mini-B, parallel, etc.) or non-standard, as desired or required.

Further, a fluid delivery module 100 can comprise one or more memory, communication and/or other types of slots or connections. Thus, the module 100 can be upgraded with additional programs, functions and/or other capabilities in accordance with a desired protocol. In some embodiments, a fluid delivery module 100 comprises a USB or other port that is configured to communicate with a personal computer, a PDA, a Smartphone and/or any other device (e.g., the hospital's computing network, an internet connection, a monitoring device, an ultrasound device, another medical device, etc.). In yet other arrangements, the fluid delivery module 100 includes one or more wireless connections or communication systems (e.g., modem, Wi-Fi, RFID, Bluetooth, etc.) that advantageously permit the module to selectively communicate with other components of the injection system and/or one or more other computing systems or devices. These types of communication devices and/or systems can permit a user to transfer data (e.g., continuously or intermittently) to and/or from the module 100, as desired or required. For example, new software or software patches can be periodically installed onto the module 100, either automatically or manually. In other embodiments, information about a particular treatment procedure (e.g., patient information, date and time, drug types, dosages and volumes injected, other injection protocol details, etc.) is selectively transmitted from the fluid delivery module 100 to an external source (e.g., network, computer, etc.).

The fluid delivery module 100 can comprise and/or can be in communication with one or more processors, control devices and/or the like. This can permit the module 100 to adequately process data and control the operation of the various components of the fluid injection/aspiration system. In some embodiments, the processor and/or control unit are included within the housing 110 of the fluid delivery module 100. Alternatively, such components can be external to the module 100. In such arrangements, the fluid delivery module 100 can be placed in data communication with an external processor and/or control unit using one or more hardwired and/or wireless communications.

According to some embodiments, a fluid delivery module and/or any other component of an injection system can be selectively configured to integrate or cooperate with one or more other devices, such as, for example, an ultrasound device or system, another type of imaging device or system and/or the like. As a result, a physician or other clinician can more accurately determine the location of the needle extending from the distal end of the syringe assembly 300 as it is being inserted into a patient's anatomy. In such arrangements, data, images and/or other information regarding the injection procedure can be provided to the user on a display of the fluid delivery module, on a display of an ultrasound or other imaging device, a separate display and/or as otherwise required or desired. Regardless of the level of integration between the injection system, an imaging device and/or another device or system, providing important data, images and other information in a single display can advantageously permit a user to more efficiency and effectively execute an injection procedure.

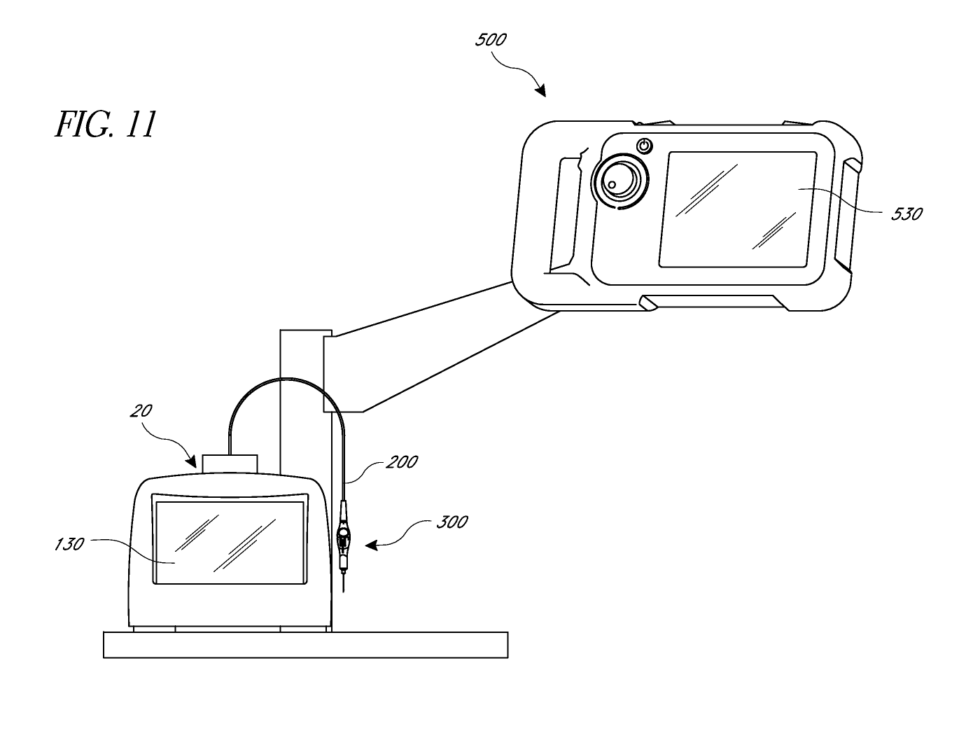

FIG. 11 illustrates one embodiment of such an integrated set-up, in which the fluid delivery module 100 is operatively connected to an ultrasound or other imaging device 500. The fluid delivery module 100 can be configured to communicate with the imaging device 500 using one or more hardwired (e.g., USB, Ethernet, other cables, etc.) and/or wireless (e.g., radio frequency, Bluetooth, etc.) connections. As shown, once operatively connected to each other, one or both of the displays 130, 530 can be configured to provide data, images and/or other information obtained by both the injection and the imaging systems. In the illustrated embodiment, the imaging system's display 530 is configured to show the status (e.g., volume of a fluid delivered, volume of a fluid remaining within the syringe, flowrate, pressure, flowrate, etc.) of the injection procedure in addition to an ultrasound image.

Thus, either or both displays 130, 530 can be configured to simultaneously provide information regarding both the imaging and the injection aspects associated with a particular procedure. Accordingly, a clinician can use a single display of the fluid delivery module or other portion of an injection system to help perform an injection procedure. Further, in some embodiments, color Doppler technology can be used to permit a clinician or other user to visualize the various steps of an injection procedure in real time. As discussed in greater detail herein, such screenshots and other images can be saved for billing, recordkeeping and/or other evidentiary purposes.

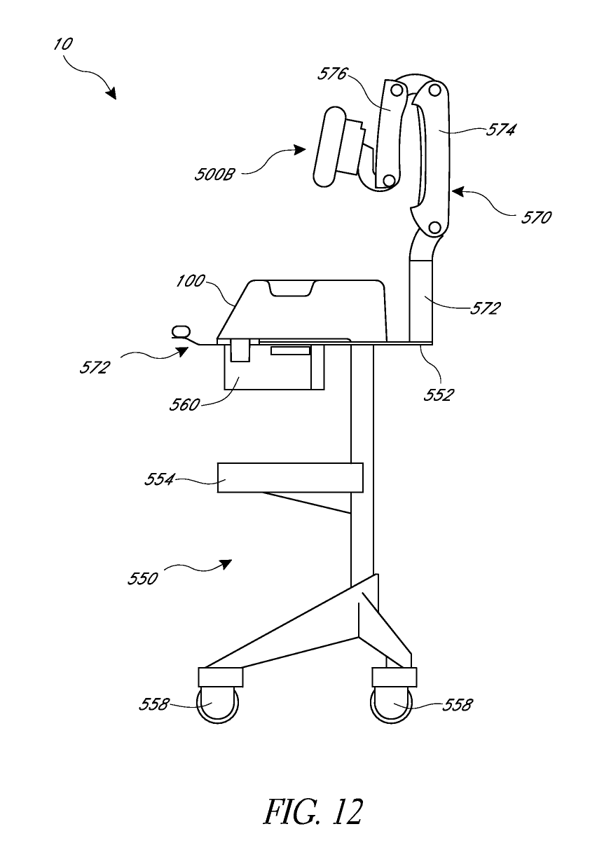

Another embodiment of an injection system 100 operatively coupled to an imaging (e.g., ultrasound) device or system 500B is illustrated in FIG. 12. In the depicted arrangement, the injection system 10 comprises a fluid delivery module 10 that can be removably positioned on a tray or other platform 552 of a cart 550. As shown, the cart 550 can include wheels 558 or other devices that allow it to be easily and conveniently moved to a desired location. In addition, the cart 550 can include one or more other trays 554, platforms and/or other features to further enhance its storage capabilities and overall functionality. The ultrasound or other imaging device 500B, which in some embodiments includes a display 530B, can also be secured to the cart 550.

As illustrated in FIG. 12, the display 530B and/or any other component or portion of the imaging device 500B can be mounted on a pivotable support member 570. In some embodiments, the support member 570 comprises one or more arms 572, 574, 576 that can be moved relative to each other (e.g., using hinges, joints, etc.) to position the display 530B and/or other portions or components of the imaging device 500B in a desired location or orientation. In some embodiments, as illustrated in FIG. 12, the cart 550 can be configured to receive a printer 560 or other output device. For example, such a printer can be used to generate a report or summary of an injection or other treatment procedure.

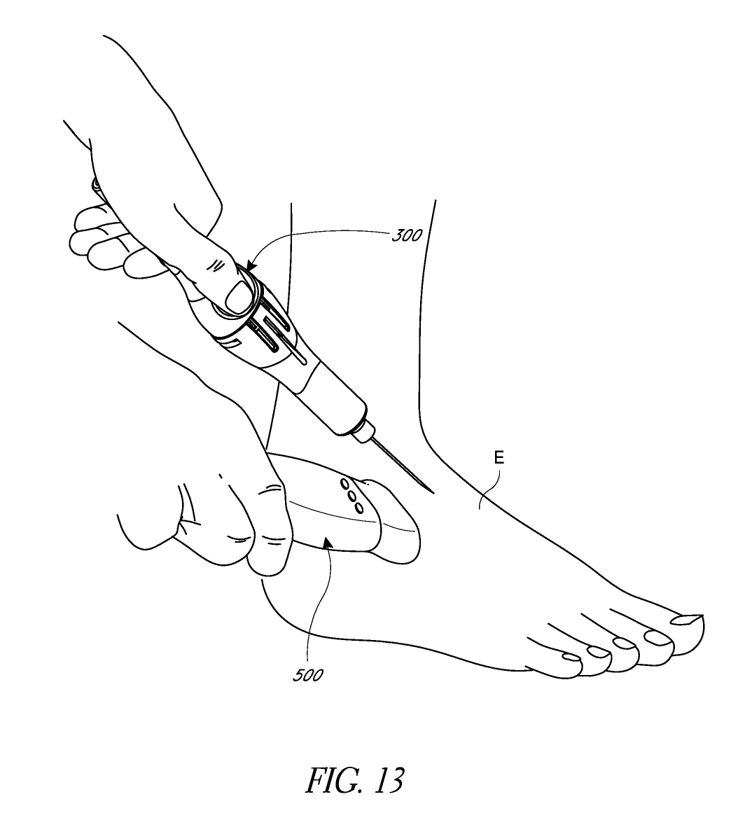

One embodiment of an ultrasound or other imaging wand configured for use with an injection system is illustrated in FIG. 13. As shown, the wand 500 can include a main body 510 and a head 514 that is configured to contact the patient's skin during the imaging procedure. In addition, the wand 500 can include one or more buttons 504, 506, 508, knobs, levers, switches and/or other controllers that allow the clinician to operate one or more aspects of the imaging system and/or the injection system. For example, the buttons and/or other controller can be configured to adjust or capture an ultrasound or other type of image. In some embodiments, the buttons are configured to regulate the injection of fluids and/or other materials through the syringe assembly 300 (e.g., initiate or terminate an injection procedure, alter the flowrate or sequence of delivery, etc.).

Accordingly, a clinician or other user can control various aspects of an injection procedure through a single device. Alternatively, the syringe assembly 300 can include one or more buttons, knobs and/or other adjustment devices or controllers that are adapted to control the delivery of a fluid and/or other material through the pre-filled syringe or other container located in the syringe assembly 300 and the operation of an imaging system, either in lieu of or in addition to button or controllers on the imaging wand. As discussed herein, this can advantageously permit a user to locate a targeted anatomical space (e.g., a joint, an organ, a cavity, etc.), control the delivery of a fluid and/or other substance to such a targeted space and/or regulate one or more other aspects of an injection procedure without having to remove his or her hands from the syringe assembly 300. In other embodiments, both the injection and imaging systems are controlled by buttons or other adjustment devices located on the fluid delivery module 100 (e.g., touchscreen display), another portion of the injection system and/or a separate device, either in lieu of or in addition to buttons located on the syringe assembly 300, the fluid delivery module 100 and/or the imaging wand 500.

The incorporation of imaging technologies (e.g., ultrasound, radio frequency spectroscopy, CT, MRI, etc.) into an articular injection system that is also configured to selectively a transfer fluid and/or other materials into or out of a targeted anatomical location can facilitate an injection/aspiration procedure for a clinician. In some embodiments, an imaging-enabled injection system can facilitate execution of a particular injection procedure. In addition, such devices and systems can enable an injection procedure to be completed with fewer clinicians and other resources. For example, when a separate imaging device is utilized, two or more physicians or clinicians are typically needed to properly and safely complete the procedure. As illustrated in the embodiment of FIG. 13, a clinician or other user can perform an injection procedure by manipulating an imaging (e.g., ultrasound, radio frequency spectroscopy, etc.) wand 500 in one hand to locate the targeted anatomical location (e.g., toe, foot, knee, other joint, etc.), while simultaneously handling the syringe assembly 300 of the injection system in the other hand to selectively transfer a fluid and/or another material to (or from) such location.

Consequently, incorporating imaging technologies into the articular injection system can offer a number of advantages. For example, such a combination unit can be operated using a single power supply. In addition, such a configuration can be operated using a single logic board, computer chip or other processor. Further, as discussed, the combination unit can allow a clinician to use “multi function” buttons and controls. For instance, one or more buttons, soft keys and/or other adjustment devices can be used to control both an ultrasound unit (or other imaging or location device) and the injection system.

As discussed, in any of the embodiments disclosed herein, a target intra-articular location or other anatomical space can be located using one or more imaging techniques, such as, for example, ultrasound, fluoroscopy, CT, MRI and/or the like. Ultrasound technology uses sound waves of a particular frequency to image internal structures (e.g., tissue, organs, joints, etc.). In some arrangements, pulsed and/or continuous sound waves can be directed to the area of interest using one or more transducers. Redirected sound waves that bounce off anatomical structures are detected by the transducers or other devices (e.g., wand 500). These data can then be processed to generate an image or other visual display of the targeted area.

Ultrasound transducers and other components used to locate a desired anatomical location can be directly or indirectly incorporated into a fluid injection system. For example, in some embodiments, a separate ultrasound probe or wand is used to visually confirm the location of the needle relative to the target location (e.g., a joint or intra-articular space, an organ, etc.). The ultrasound equipment can be configured to operate either continuously or intermittently during the course of the procedure, as desired or required. In other embodiments, an ultrasound transducer (or another ultrasound device) is incorporated directly into one or more components of an injection system. For instance, a small ultrasound transducer can be positioned at or near the tip of the delivery or aspiration needle. The ultrasound transducer can be placed in data communication with a processing apparatus and/or other components using one or more hardwired and/or wireless connections. In addition, the injection system can be configured so that the imaging results are advantageously viewed on the display of the fluid delivery module.

Thus, as the needle is inserted into the body, a physician or other clinician can accurately detect the position of the distal end of the needle. Such imaging techniques can be used alone or in conjunction with one or more other locating methods or devices. For example, in one embodiment, tissue response measurements can be used to locate a target intra-articular space. In other embodiments, ultrasound and/or other imaging technologies are used to locate a targeted intra-articular space. In other embodiments, both tissue response measurements and ultrasound and/or other imaging technologies are used to locate a joint space. In still other embodiments, one or more other joint locating methods or devices can be used, either in lieu of or in addition to methods, systems and methods disclosed herein.

In some embodiments, ultrasound imaging is particularly advantageous because it permits real-time visualization of a joint or other target location. By way of example, in one embodiment, the delivery module and system include an ultrasound device using a broadband curved array transducer working at about 2-5 MHz and a broadband linear array working at about 4-7 MHz. Imaging errors can be kept at a minimum by taking the linear array for measurements. Curved array may be desirable and used for better penetration depth.

Several embodiments of the present application provide a system and method of using ultrasound guidance to inject fluids into small joint spaces. Further, ultrasound and other imaging technologies can assist in the visualization of internal structures (e.g., bones, joints, organs, other tissue, etc.) within the anatomy. Thus, such imaging technologies can be used to visually display the orientation of the needle with respect to such internal structures. Consequently, ultrasound can assist a user in correctly positioning and directing the needle during an injection and/or aspiration procedure.

In addition, a contrast media can be used with the ultrasound devices and methods described herein to further enhance the user's ability to verify the location of the needle tip relative to the targeted anatomical location (e.g., intra-articular location, organ, etc.). This can provide additional assurances that the medication, other fluid and/or other substances are being delivered to the desired location within the patient being treated. A contrast media can also be used in embodiments where aspiration of a fluid or other material is desired. For example, if acceptable, a contrast media can be delivered to or near the desired location. Then, once placement of the aspiration needle has been confirmed, the fluid module can be used to aspirate as required. In some embodiments, if the aspiration procedure is therapeutic in nature (e.g., being used to relieve pressure within the targeted anatomical location), the use of contrast media may be acceptable. However, in one or more other circumstances, the use of contrast media may not be acceptable or desirable. For example, if the purpose of the aspirating is to withdraw a fluid for diagnostic reasons (e.g., testing the extracted fluid sample), initially injecting a contrast media or other substance may contaminate the desired sample.

As discussed herein, in some embodiments, data and other information regarding the types, volumes or other amounts, dosages and/or other details of the various medications and/or other substances administered during a particular injection procedure, as displayed to the user in a touchscreen or other interface, are automatically stored within a memory of the fluid delivery module, another component or portion of the injection system or an external processor or network with which the injection system is in data communication. In addition data and information related to ultrasound or other imaging procedures that were conducted can also be saved for later processing (e.g., documentation, billing, etc.) or retrieval. Such data and information can include actual ultrasound images, details regarding the imaging equipment used, the extent to which a particular imaging device was used and/or the like.

In addition, as discussed, other details related to a specific procedure can also be recorded, maintained and linked to a delivery sequence of various medicaments and/or other substances. For example, the injection system can be configured to receive and maintain the name of the patient, the date and time that the procedure was performed, the duration of the procedure, the physicians, clinicians and/or other personnel that participated in the preparation and/or execution of the procedure, the disease or condition being treated, specific treatment codes and other administrative information and/or the like. Such data collection capabilities can assist with billing, insurance processing, patient record keeping, generation of reports, reordering of medicaments and other injectable materials and/or other functions. In some embodiments, such records or summaries (e.g., printouts, electronic file, etc.) can be included in or otherwise connected with (e.g., physically, electronically, etc.) a patient's file or chart. In addition, the use of the summaries or reports can provide one or more additional benefits to a user. For example, such summaries and reports can improve the economic return on an injection procedure for the service provider by leveraging the relatively favorable reimbursement of the corresponding ultrasound-guided (or other imaging-guided) procedures.

According to some embodiments, an injection system includes a printer, another output device, memory and/or the like to help memorialize the details associated with a specific injection procedure. As noted herein, the corresponding output resulting from such recordkeeping can assist with billing, insurance processing, patient record keeping, generation of reports and/or the like. In addition, such printouts or alternative forms of output (e.g., electronic reports) can memorialize the details of a particular procedure, serving as evidence of what was performed (e.g., which and how much of each medicament and/or other substance was injected, the sequence of delivery, visual confirmation via an ultrasound or other imaging technology of the needle location and other details of the injection, etc.), to whom the injection was administered, who performed the injection procedure, when and where the procedure was executed and/or the like. As noted above, such summaries can be provided on a paper printout (e.g., a printer that is incorporated with or operatively coupled to an injection system), electronic form (e.g., a summary generated as a pdf, an image or some other standard or non-standard viewable format, etc.) and/or the like.

Reports or summaries generated by printer or another output device of the system can comprise paper printouts, electronic files and/or the like, as desired or required. In some embodiments, the report or summary includes one or more images of the patient's joint or other targeted anatomical location. Such images can be generated using ultrasound or other imaging technologies. In some embodiments, the images provide visual confirmation of the location of the injection system's needle in relation to the patient's anatomy. In addition, the images can provide details related to the injection of fluids and/or other materials to or near the targeted joint or other anatomical location. For example, Doppler or other technologies can be used to verify that the various fluid and/or other material streams were properly delivered to the patient.

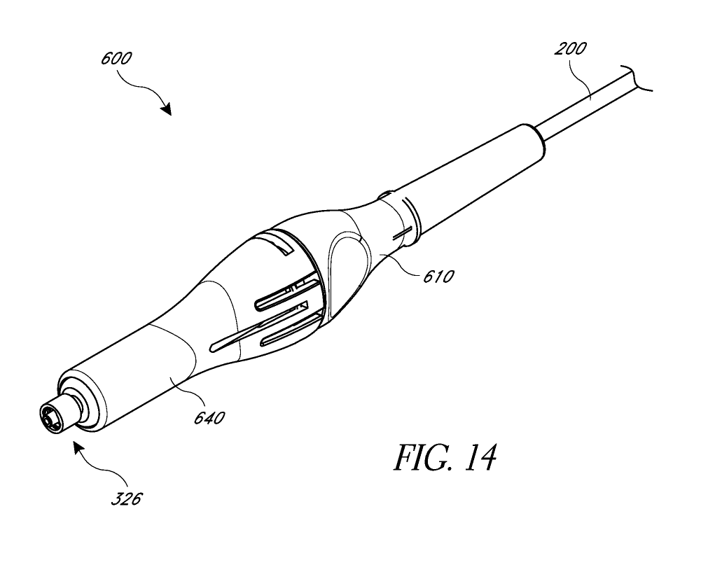

With reference to FIGS. 14-17, any injection system disclosed herein can comprise a handpiece assembly to facilitate handling and/or manipulation of a pre-filled syringe. As shown, the handpiece assembly 600 can comprise a proximal handle 610 and a distal adapter 640. Thus, the handpiece assembly 600 can include two or more portions 610, 640 that are configured to removably secure to one another. Alternatively, the handpiece assembly 600 can comprise a unitary structure.

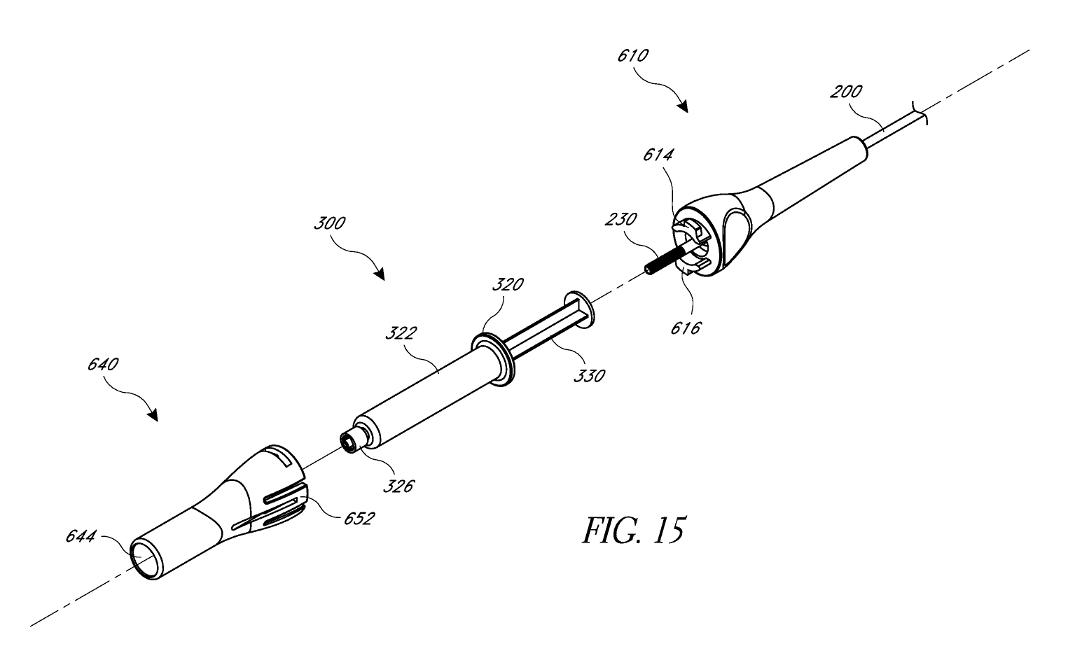

As illustrated in FIG. 15, the handle 610 can comprise an internal passage that is sized, shaped and otherwise adapted to receive a motion transfer cable 200 (e.g., push/pull cable). For example, in some embodiments, the distal terminal 230 of the cable 200 can extend through a passage or other opening of the handle 610. The terminal 230 can be configured to quickly attach to and detach from the plunger 330 of the syringe assembly 300. For example, the terminal 230 can include a threaded end and/or other quick-connect type attachment feature. As discussed herein with reference to other embodiments, the connection between the motion transfer cable 200 (e.g., terminal 230, other portion of the cable, etc.) and the syringe assembly 300 can vary, as desired or required.

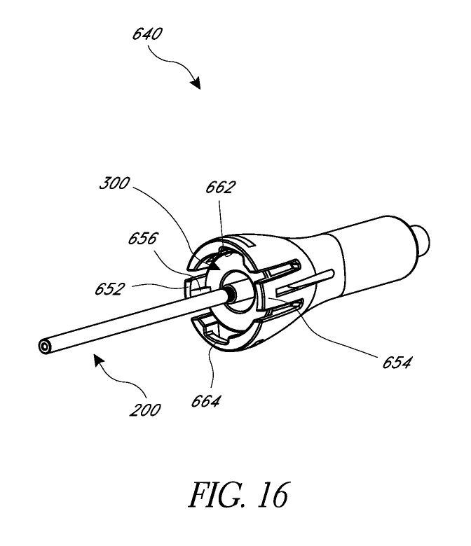

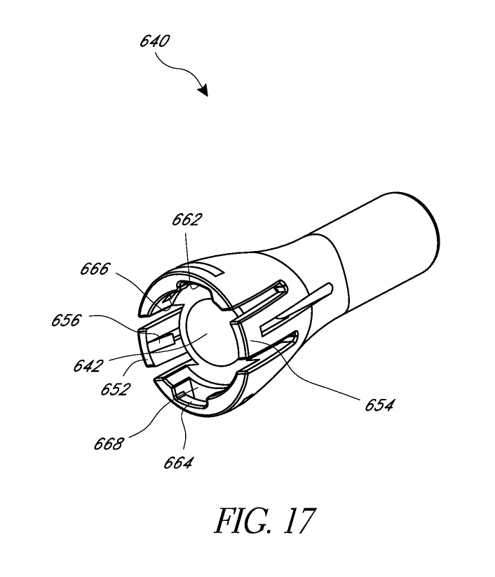

With continued reference to FIGS. 16 and 17, according to some embodiments, the adapter 640 is configured to slidably receive a syringe assembly 300. For instance, as shown, an internal cavity 642 of the adapter 640 is sized and shaped to accommodate and retain a pre-filled syringe 300 or other container. In addition, as illustrated in FIGS. 14 and 15, the adapter 640 can comprise a distal opening 644 through which a distal, discharge end 326 of the syringe assembly 300 may extend. As discussed herein, the distal end 326 of the syringe assembly 300 can include a luer connection or other standard or non-standard fitting (e.g., for removably attaching to a needle assembly).

According to some embodiments, the distal adapter 640 comprises one or more snaps, tabs or other members 656 that are adapted to engage a portion of the syringe assembly (e.g., a flange portion of the syringe barrel) and/or otherwise help retain the syringe assembly within the cavity 642 of the adapter 640. In some embodiments, the adapter 640 is disposable so that the syringe assembly 300 (e.g., the pre-filled syringe) is discarded together with the adapter 640 after use. Alternatively, however, the adapter 640 can be reusable, requiring a syringe assembly 300 to be removed from the adapter after a desired amount of the syringe assembly 300 has been discharged. In such embodiments, the adapter 640 can be cleaned, sterilized (e.g., autoclaved), disinfected and/or otherwise treated between uses and/or over time, as desired or required. Accordingly, the handpiece assembly 600 can comprise one or more materials, such as, for example, polymeric materials, metals or alloys, other natural or synthetic materials and/or the like.

With continued reference to FIGS. 15-17, once the syringe assembly 300 has been properly inserted and/or secured within the adapter 640, the handle 610 can be coupled to the adapter. In the depicted embodiment, the handle 610 comprises a pair of tabs or other protruding members 614, 616 that are sized, shaped and otherwise configured to mate with corresponding recesses or openings 662, 664 of the adapter 640. In one embodiment, once the tabs 614, 616 have been aligned with and inserted into the corresponding recesses 662, 664, the handle 610 is rotated or otherwise turned relative to the distal adapter 640 in order to secure the handle to the adapter. Thus, each of the recesses 662, 664 of the adapter 640 can comprise radially-oriented slots 666, 668 in which at least a portion of the tabs 614, 616 can selectively move and secure.

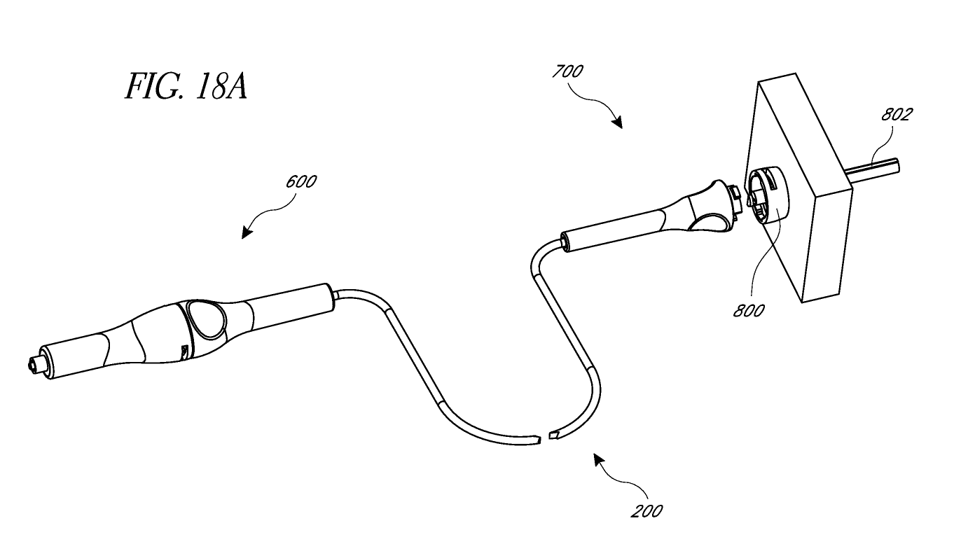

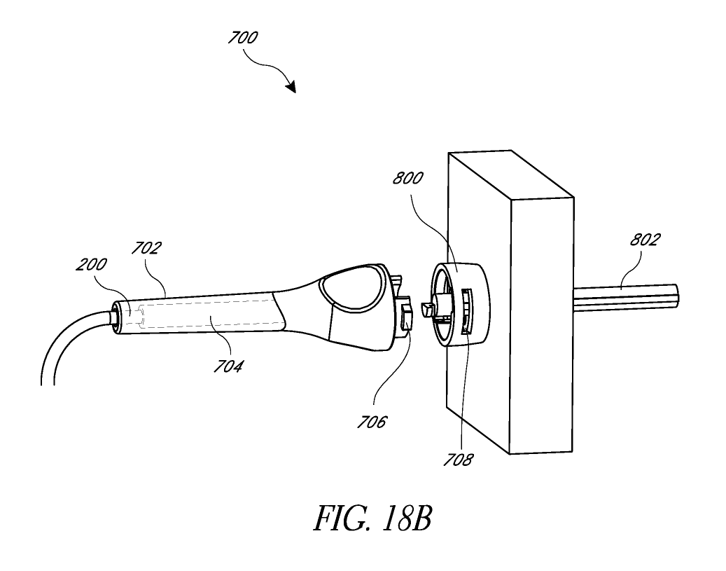

FIGS. 18A and 18B illustrate another embodiment of an injection system that utilizes a motion transfer cable to accurately discharge fluids and/or other materials from a syringe or other reservoir. As with other embodiments disclosed herein, the depicted system comprises a handpiece assembly 600 and a motion transfer cable 200. The illustrated embodiment also comprises a cable connector 700. As discussed herein, the handpiece assembly 600 can be configured to removably or permanently receive a syringe assembly 300 therein. However, in other arrangements, the injection system comprises a syringe assembly 300 without a handle or other portions or components of a handpiece assembly 600, as desired or required.

The cable connector 700 can be attached to the proximal end of the motion transfer cable 200, generally opposite of the syringe assembly. In some embodiments, such as in the system illustrated in FIGS. 18A and 18B, the cable connector 700 comprises an outer housing 702 surrounding an inner cable shaft 704, and the proximal end of the motion transfer cable 200 can be attached (e.g., fixedly or removably) to the cable shaft 704. A proximal end of the cable connector 700 can be configured to selectively attach to and detach from a motor assembly via a motor unit receptacle, coupling, fitting or other feature 800. As shown in FIGS. 18A and 18B and discussed in greater detail herein, in some embodiments, the cable connector 700 is sized, shaped and otherwise adapted to mate with a corresponding portion of a motor unit receptacle 800. For example, the cable connector 700 can be configured to rotatably fit within and mate to a corresponding portion of the motor unit coupling, receptacle, fitting or other engagement site 800. In the depicted embodiment, the motor unit receptacle 800 comprises an outer ring or coupling located on, along or near a motor assembly (e.g., a motor, an actuator, a drive shaft and/or any other movable device). The actuator, drive shaft or other output device or member 802 of the motor assembly can directly or indirectly engage (e.g., releasably attach, couple, etc.) the motion transfer cable 200, for example, via inner cable shaft 704, and help move the motion transfer cable 200 relative to the syringe assembly.

The adjacent mating portions of the cable connector 700 and the motor unit receptacle or coupling 802 can include a tab and a corresponding slot into which the tab can be inserted and rotated or otherwise moved (e.g., to couple the cable connector to the actuator or other output member of the motor assembly). However, in other embodiments, one or more other mating features or methods can be used to releasably secure the motion transfer cable to an output end of a motor assembly, such as, for example, threaded connections, friction fit connections, snap-on connections and/or the like.

As discussed above, in some embodiments, the proximal end of the cable connector 700 is configured to releasably engage a motor assembly via a motor unit receptacle or coupling 800 (or other quick-connect fitting) to advantageously allow for fast connection and disconnection of the motion transfer cable from the motor assembly. For example, in one embodiment, when the proximal end of the cable connector 700 and motor unit receptacle 800 mate, tabs 706 on the proximal end of the cable connector 700 engage corresponding recesses or openings 708 on the motor unit receptacle 800 in a lock-key manner. Thus, when such mating portions are aligned and engaged, one or more features of the cable shaft 704 and corresponding features on the drive shaft 802 can align and engage. However, the motion transfer cable can be coupled to an output end of a motor assembly (e.g., an actuator, a drive shaft, etc.) without the use of a cable connector and/or other quick-connect features.

Once the motion transfer cable 200 has been properly secured to an output member 802 (e.g., drive shaft, actuator, other mechanical linkage or movable member, etc.) of a motor assembly, the output member 802 can advantageously move (e.g., distally or proximally) the motion transfer cable 200 to as to discharge (or fill) a syringe assembly 300 coupled to the distal end of the cable 200. Thus, the quick-connect features described herein can advantageously allow for faster and easier handling of the motion transfer cable 200. For example, the need may arise to change the cable 200 from a lighter duty cable to a heavier duty cable (e.g., for higher density or viscosity materials, such as hyaluronic acid, steroids, bone cement, etc.), or vise versa. Such quick-connect features can also provide one or more other benefits and advantages, such as, for example, easier sterilization and cleaning between or during injection procedures, easier and more convenient repairs and maintenance and/or the like.

In some embodiments, the motor unit receptacle or coupling 800 is connected to or incorporated into a fluid delivery module, such as, for example, the module 100 disclosed herein. Alternatively, the coupling 800 can be connected to or incorporated into another device, such as, for example, a separate motor (e.g., stepper motor), an actuator and/or any device configured to accurately and adequately move the motion transfer cable against the back pressures that may be experienced during an injection or aspiration procedure. In some embodiments, the motor, actuator or other device can be secured (e.g., at least temporarily) and/or otherwise placed on a table or cart in the procedure room, the bed, an adjacent wall and/or the like. Alternatively, the motor, actuator or other device can be attached to the physician or other clinician (e.g., the physician's arm, waist, etc.), as desired or required. In one embodiment, for example, an actuator or other movable member is powered by a stepper motor and comprises a range of motion of about two to six inches (e.g., 2, 3, 4, 5, 6 inches, values between the foregoing lengths, etc.). In other arrangements, however, the range of motion can be greater than 6 inches or less than 2 inches, as desired or required by a particular application or use.

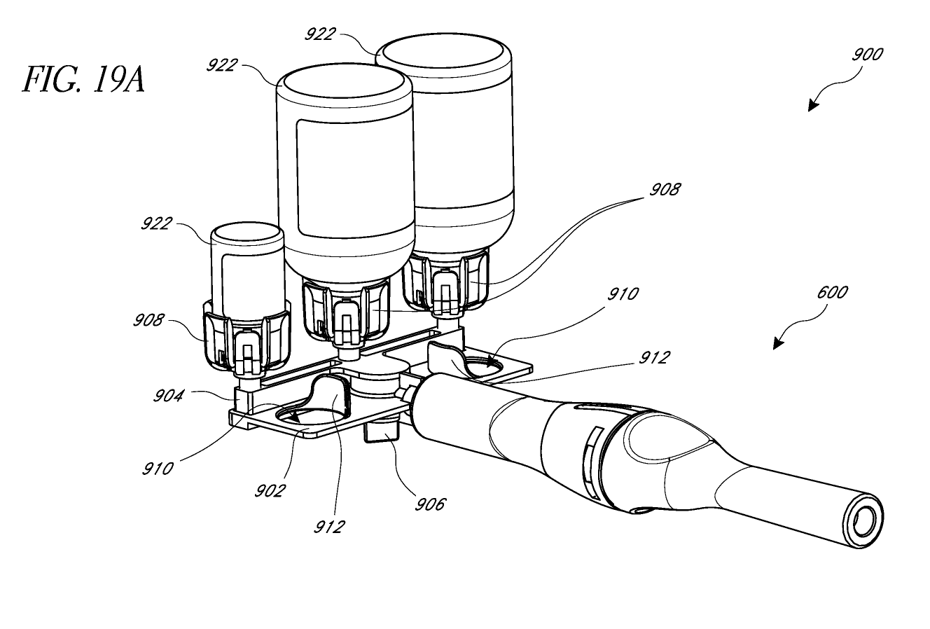

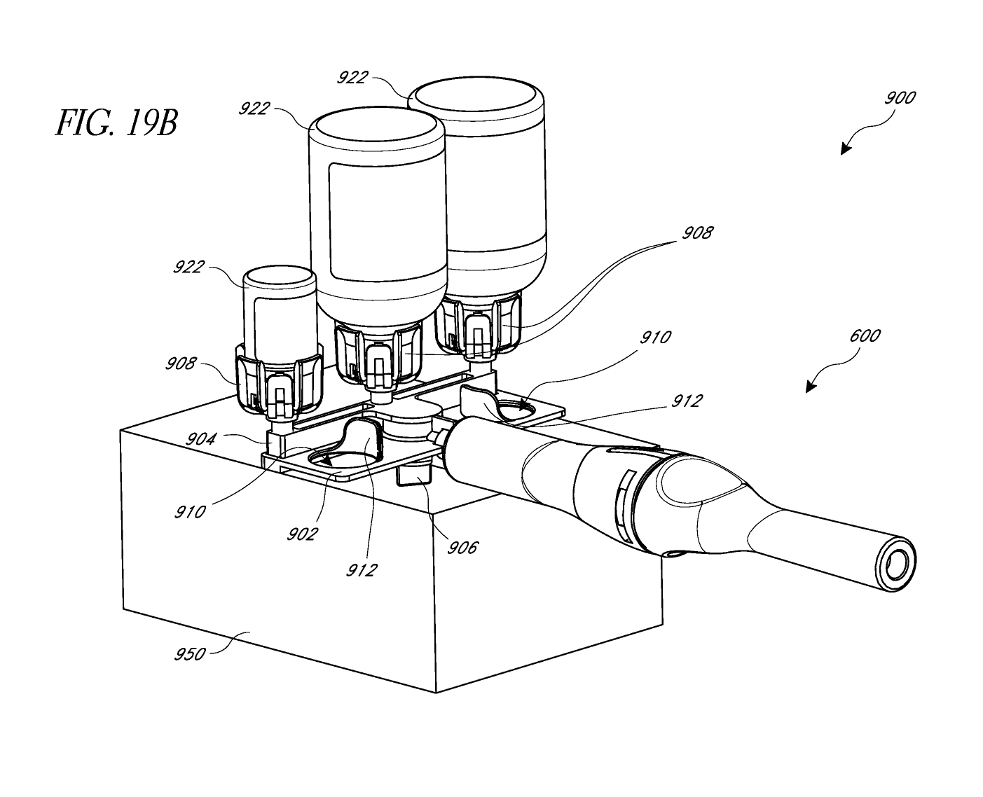

FIG. 19A illustrates an example embodiment of a filling system 900 for filling a syringe with one, two or more different fluids and/or other medicaments. Such a filling system can facilitate and expedite the filling of pre-filled syringes or other containers that are placed in fluid communication with the filling system. For example, the filling system can be used to transfer, in a sterile and accurate manner, predetermined volumes of one or more fluids and/or other medicaments from corresponding vials or other containers to a syringe. As discussed in greater detail herein, according to some embodiments, the filling system can be operatively coupled to a fluid delivery module, an actuator, a motor and/or other device that can help at least partially automate the transfer of fluids from the vials (or other containers) to a syringe.

As illustrated in FIG. 19A, in some embodiments, the system 900 comprises a base 902, a manifold 904 and a valve or fluid selector 906. As shown, one or more vial connectors 908 can be used to facilitate securing the various vials and/or other container 922 to the system 900. In some embodiments, the containers 922 that are secured to the filling system 900 comprise standard or non-standard vials as provided by a medicament manufacturer or supplier. Vial connectors 908 or similar devices can advantageously enable the system 900 to receive vials and/or other container 922 of various sizes, shapes and/or configurations.

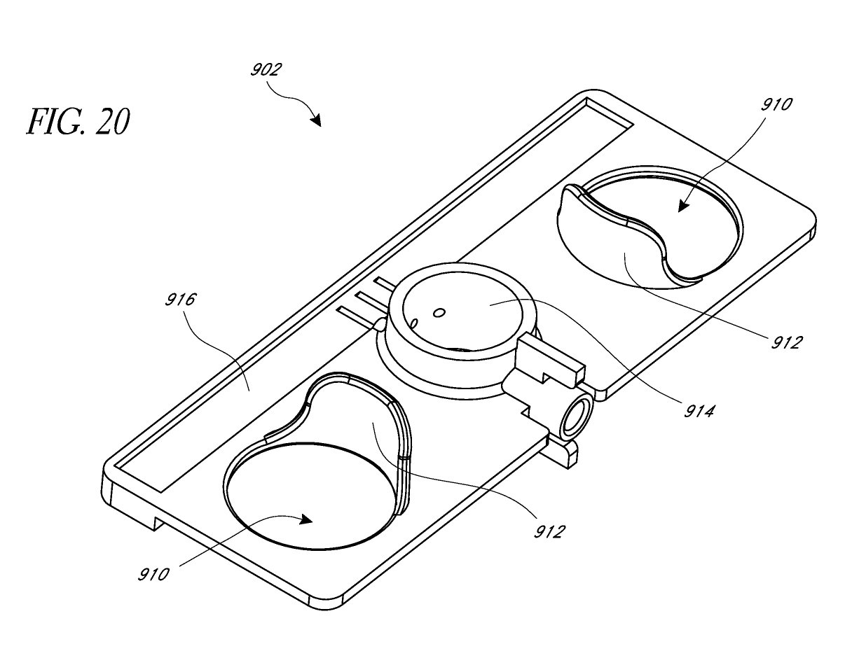

With reference to FIG. 20, the base 902 can be generally rectangular and can have a circular opening 910 on each lateral side or edge. However, in other embodiments, the base can comprise any other shape (e.g., oval, circular, other polygonal, etc.), arrangement or configuration. As shown, one or more flanges or tabs 912 can extend upwardly from the inner edges of the circular openings 910. Such flanges or tabs can facilitate in the grasping and manipulation of the base 902 (and any components of the filling system coupled to the base). Further, with further reference to FIG. 19A, the valve or fluid selector 906 of the filling system 900 can be sized, shaped and otherwise configured to fit into a cylindrical housing, recess, opening or other portion 914 formed in or near the center of the base 902. The valve 906 can be secured within the cylindrical housing by a friction fit or any other connection or method.

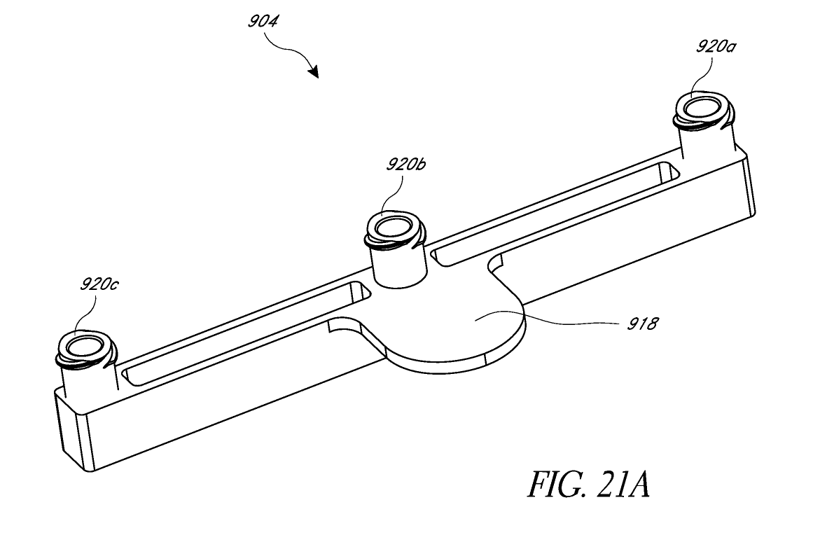

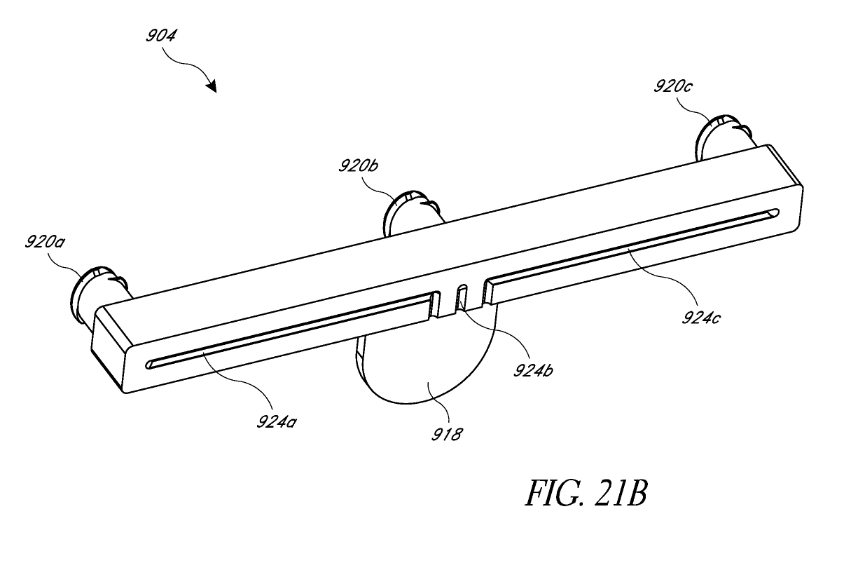

FIGS. 21A and 21B illustrate a manifold 904 of the filling system 900. As shown in FIGS. 19A and 19B, the manifold 904 can be secured into a recess 916 on or along one or more surfaces or portions of the base 902. In some embodiments, the manifold 904 is secured to the base 902 by ultrasonic welding that extends either partially or completely around the perimeter of the manifold 904. However, any other type of connection or attachment device or method can be used, either in lieu of or in addition to welding, such as fasteners, adhesives and/or the like. In some embodiments, the manifold 904 includes a flange 918 that extends forwardly and covers, at least partially, the top of the valve 906 when the filling system 900 is assembled. One or more inlet ports or container receiving sites 920a, 920b, 920c can extend upwardly from the manifold 904. As discussed in greater detail herein, such inlet ports or receiving sites can be adapted to directly or indirectly (e.g., using a vial connector or similar device) receive a vial or container. The illustrated embodiment comprises a total of three inlet ports, nests or connection sites, including a central inlet port 920b and side inlet ports 920a, 920c along each lateral side or edge of the manifold 904. The inlet ports 920a, 920b, 920c and/or the accompanying vial connectors 908 that are adapted to secure to the ports can be configured to removably receive vials or other containers 922 (FIG. 19A) that contain fluids and/or other medicaments. As noted above, such vials or containers can comprise a variety of shapes, sizes and/or other configurations. According to some embodiments, in order to facilitate the connection of the vial connectors 908 to the ports or connection sites 920a, 920b, 920c of the manifold, the inlet ports or connection sites of the filling system 900 comprise a standard or non-standard connection or fitting, such as, for example, a luer fitting.

With reference to the embodiment illustrated in FIG. 21B, the manifold 904 and other portions of the filling system 900 can include one or more fluid channels that place one or more of the inlet ports (and thus, the vials or containers secured thereto) in fluid communication with an outlet port 936 of the filling system 900. For example, in the depicted arrangement, the manifold 904 comprises three fluid channels, 924a, 924b, 924c, each of which extends generally vertically from a corresponding inlet port or connection site (e.g., luer fitting) 920a, 902b, 920c to the bottom or lower portion of the manifold 904. As shown, these fluid channels then extend generally horizontally along or near the bottom of the manifold 904. In the depicted arrangement, the horizontal portion of channel 924b that is in fluid communication with the central inlet port or connection site 920b travels from the center to the front of the recess 916 in the base 902. Likewise, the horizontal portions of channels 924a and 924c originating from the side inlet ports or connection sites 920a and 920c can travel from the periphery of the recess 916 in the base 902 inwardly toward channel 924b, and then extend generally parallel to channel 924b from the center to the front of the recess 916. In some embodiments, the fluid channels 924a, 924b, 924c extend forwardly from the recess 916 through the main body of the base 902 and the valve case 914. As shown in the embodiment illustrated herein, a base fluid channel 932 can extend through the base 902 from the front of the valve case, recess or other opening 914 to or near the front of the base 902.

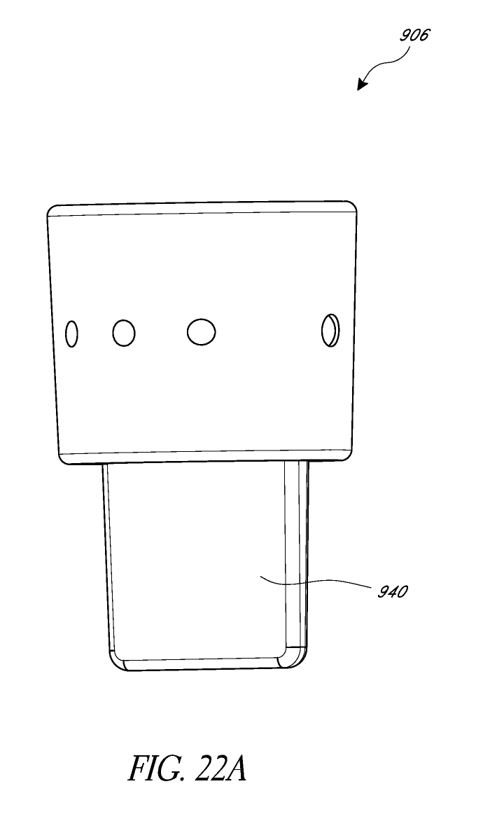

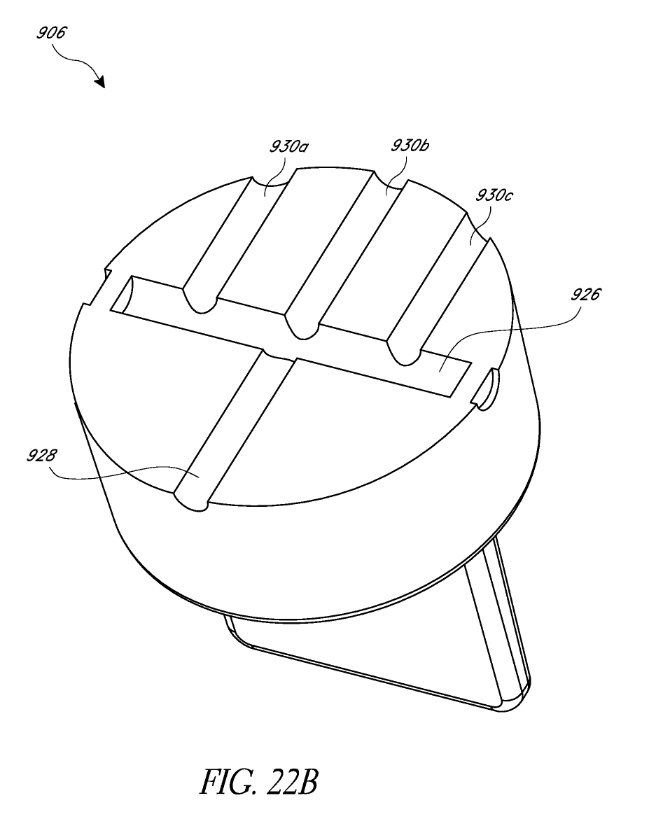

With reference to FIG. 22A, the main body of the valve or fluid selector 906 can be generally cylindrical. However, in other embodiments, the valve or fluid selector can comprise any other shape (e.g., oval, rectangular, other polygonal, irregular, etc.) or general configuration, as desired or required. As shown in the cross-sectional view of FIG. 22B, the valve 906 can comprise a central fluid channel 926 that extends at least partially across the valve. Further, the valve or fluid selector 906 can include an entrance fluid channel 928 running from or near the rear of the valve 906 to the central fluid channel 926, and three exit fluid channels, 930a, 930b, 930c, which, in the depicted embodiment, are generally parallel to one another and run from the central channel 926 to the front of the valve 906. In some embodiments, the exit fluid channel 930b is generally in-line with the entrance channel 928. However, in other embodiments, the exact configuration, orientation, spacing, size, shape and/or other details of the fluid channels and/or other portions of the valve can vary.

Regardless of its exact configuration and design, the valve or fluid selector 906 can be configured to be moved between two or more positions in order to selectively place one of the various inlet ports (and thus the interior contents of a vial or other container attached to such a inlet port) in fluid communication with the outlet port 936 of the filling system 900. Thus, in other embodiments, a different type of valve (e.g., a two-way, three-way, four-way, etc.) can be used to allow a user to manually or automatically deliver fluids and/or other medicaments from one or more vials or other containers secured to the filling system to a syringe.

According to some embodiments, the valve or fluid selector 906 comprises a downwardly extending tab 940 configured to engage, for example, an actuator, a motor, solenoid and/or any other movable member or portion. Such an actuator, motor and/or other movable device can be configured to rotate or otherwise move the valve 906 (e.g., about a central vertical axis). In some embodiments, where the valve or fluid selector comprises a different configuration (e.g., is not rotatable or uses another mechanism to select a particular fluid path), the actuator or other movable device can be adapted to change a position of the valve in a different manner (e.g., slidably along a different axis, vertically, etc.).

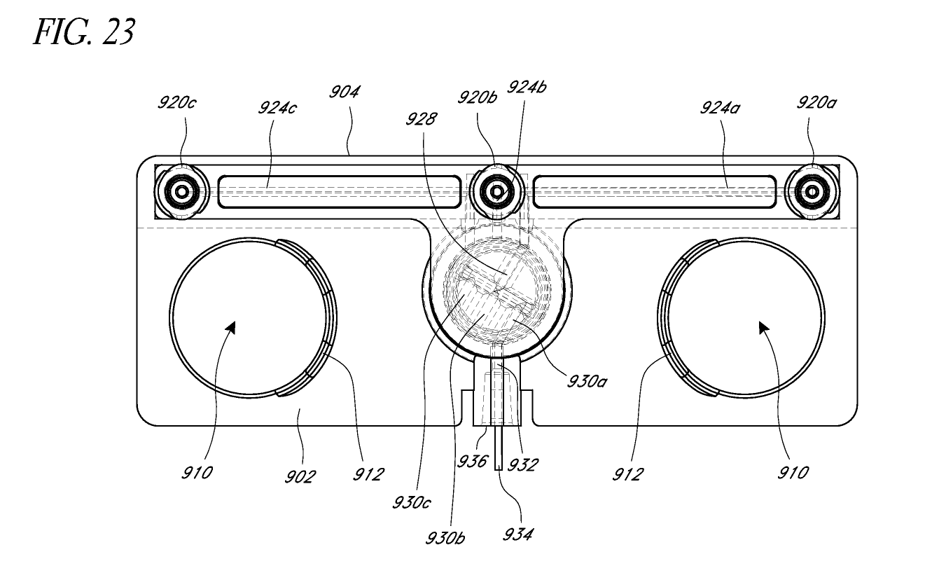

According to some embodiments, the valve or fluid selector is configured to be rotated or otherwise moved between a plurality of positions during the operation and use of the filling system 900. For instance, as shown in the depicted embodiment, the valve 906 can comprise a total of four positions to which it may be moved during use. However, as discussed herein, the valve or other fluid selector can include more (e.g., five, six, more than six, etc.) or fewer (e.g., two or three) positions to which it may be moved. By way of example, in one position, as shown in FIG. 23, the entrance fluid channel 928 is aligned and placed in fluid communication with the inlet port channel 924a, and the exit channel 930a of the valve 906 is aligned and placed in fluid communication with the base fluid channel 932. In another valve position, the entrance fluid channel 928 is aligned and placed in fluid communication with the inlet port channel 924b, and the exit channel 930b of the valve 906 is aligned and placed in fluid communication with base fluid channel 932. In a third valve position, the entrance fluid channel 928 is aligned and placed in fluid communication with the inlet port channel 924c, and the exit channel 930c of the valve 906 is aligned and placed in fluid communication with base channel 932. Finally, the core 906 can be rotated or otherwise positioned so that the entrance fluid channel 928 is not aligned or in fluid communication with any of the inlet port channels 920a, 920b, 920c. In such a position, the outlet port 936 is not in fluid communication with any of the inlet ports of the filling system 900. In other embodiments, the valve or other fluid selector can comprise more or fewer positions, depending, for example, at least in part, on the number of inlet ports, the number and configuration of any fluid channels of the system and/or the quantity, configuration and/or other properties of one or more other components of the filling system 900.

With continued reference to FIGS. 22B and 23, the base channel 932 can be configured to be in fluid communication with an outlet port (e.g., a luer fitting) 936. In some embodiments, as shown in FIGS. 19A and 19B, the outlet port is configured to removably couple, either directly or indirectly (e.g., via a fluid conduit or other fluid passage), to a distal end (e.g., a luer connection or other outlet 326) of a syringe 320 or other container. Therefore, in order to fill the syringe 320 or other container, the plunger 330 or other moveable member of the syringe or other container is retracted (e.g., generally away from the syringe's outlet 326), creating a negative pressure or vacuum in the syringe 320. If the valve 906 is positioned such that the entrance channel 928 is aligned with one of the inlet port channels 920a, 920b, 920c, negative pressure or vacuum created within an interior of the syringe 320 or other container helps to draw fluid from the corresponding vial or other container secured to the inlet port (e.g., or vial connector attached thereto) through the corresponding fluid channels (e.g., inlet fluid channel, entrance channel, exit channel and base channel) and ultimately into the syringe 320. If the valve 906 is positioned such that the entrance channel 928 is not aligned with any of the fluid channels, no fluid can flow into the syringe 320. With continued reference to FIG. 23, the valve 906 can be positioned so that entrance fluid channel 928 is aligned and in fluid communication with one of the inlet port channels 924a. This will allow for fluid flow 934 from the targeted inlet port, through the corresponding fluid channels, and through the outlet port 936 (e.g., luer fitting or other standard or non-standard coupling, fitting or connection).

In some embodiments, the syringe 320 or other container can be filled prior to being inserted into a handpiece assembly 600 and/or prior to coupling the syringe to an injection system comprising motion transfer cable 200 (e.g., in accordance with the various arrangements disclosed herein). In such embodiments, the plunger 330 or other movable member operatively coupled to an interior of the syringe or other container can be retracted manually to create the negative pressure or vacuum to fill the syringe. Alternatively, the syringe 320 can be inserted into a handpiece assembly 600 and/or otherwise coupled to the motion transfer cable 200 (e.g., in accordance with the various embodiments disclosed herein) before filling. In such embodiments, the plunger 330 can be withdrawn or retracted with the assistance of the motion transfer cable 200 to create the negative pressure or vacuum, for example, as described herein for withdrawing bodily or other fluids from a patient's anatomy. This can be particularly helpful when the syringe or other container is being filled with one or more viscous or concentrated fluids or other materials. In some embodiments, the syringe or other container can be directly coupled to a vial or other container rather than to filling system 900. The motion transfer cable 200 can then be used to draw fluid directly into the syringe or other container from the vial or other container.

As shown schematically in FIG. 19B, the base 902, manifold 904 and valve 906 assembly can be installed on a device 950 that is advantageously configured to rotate the valve 906 between its various positions. The circular openings 910 along or near the side of the manifold's base 902 can aid in the installation of the assembly on the device 950. Further, in some embodiments, as noted above, the flanges 912 extending from the circular openings 910 can provide a place for a user to grasp and manipulate the assembly (e.g., to install and remove it). The device 950 to which the valve or other fluid selector 906 is coupled can comprise a fluid delivery module 100, a simple motor, another device comprising a motor, any other type of actuator, a solenoid and/or the like.

In some embodiments, the filling system 900 (or a portion thereof, e.g., the base 902, manifold 904, valve assembly, etc.) is operatively coupled to the same fluid delivery module 100 that receives cassette 20 (see, for example, the fluid delivery modules disclosed herein with reference to FIGS. 1-17 and the fluid delivery modules disclosed in the applications that are incorporated by reference herein). Thus, the valve or fluid selector 906 can be rotated by a separate motor of the fluid module or the same motor of the fluid module that is used to selectively move the motion transfer cable 200. Therefore, in some embodiments, the motor or other movable device that is adapted to move rotate or otherwise move the valve can also provide the necessary linear movement to a motion transfer cable 200. Alternatively, the filling system 900 can be installed in and/or operatively coupled to a second fluid delivery module, another motor, a solenoid device and/or any other type of device configured to provide the necessary movement to the valve or fluid selector.

According to some embodiments, the fluid delivery module 100 controller can control the withdrawal of the motion transfer cable 200, as well as the rotation and position of the valve 906 for filling of the syringe. The controller can operate under a preset program and/or any other set of parameters entered into the system by a user. The program or parameters can advantage control the transfer of fluids from one or more vials or other containers positioned along the inlet ports of the filling system to the syringe. Thus, the types, volumes and/or characteristics of the fluids and/or other medicaments that are transferred to a syringe can be customized and carefully regulated. Accordingly, a desired or targeted fluid and/or medicament combination can be created.

To assist in the description of the disclosed embodiments, words such as upward, upper, bottom, downward, lower, rear, front, vertical, horizontal, upstream, downstream have been used above to describe different embodiments and/or the accompanying figures. It will be appreciated, however, that the different embodiments, whether illustrated or not, can be located and oriented in a variety of desired positions.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

What is claimed is:

1. An injection system for transferring a volume of fluid from a pre-filled syringe into a patient, comprising:
a syringe assembly comprising a syringe adapter configured to securely receive a syringe and a plunger assembly configured to be selectively moved relative to the syringe adapter;
at least one motor assembly configured to impart a force on and move an actuator in at least one direction;
a motion transfer cable comprising a first end and a second end and an inner core extending between said first end and said second end;
wherein the first end of the motion transfer cable is mechanically coupled to the actuator;
wherein the second end of the motion transfer cable is mechanically coupled to the plunger assembly of the syringe assembly;
wherein movement of the actuator by the motor is transferred through the motion transfer cable to the plunger assembly, resulting in the plunger assembly moving a desired distance relative to the syringe secured to the syringe assembly;
wherein fluid exiting the syringe as a result of movement by the actuator, the motion transfer cable and the plunger assembly passes through a distal outlet of the syringe; and
wherein the syringe assembly is configured to be grasped and manipulated by a clinician during an injection procedure.

2. The injection system of claim 1, wherein the at least one motor assembly is operatively coupled to a fluid delivery module.

3. The injection system of claim 1, wherein the injection system is configured to receive instructions for moving the actuator and transferring a volume of fluid out of the syringe.

4. The injection system of claim 1, wherein the actuator is mechanically coupled to a cassette that is removably coupled to the at least one motor assembly.

5. The injection system of claim 1, wherein the motion transfer cable is generally flexible to permit the syringe assembly to be manipulated by a clinician during an injection procedure.

6. The injection system of claim 1, wherein the at least one motor assembly is positioned on a surface and is configured to not move relative to said surface while the syringe assembly is used during an injection procedure.

7. The injection system of claim 1, wherein the at least one motor assembly is positioned on at least one of a cart, a table, a desk, other platform, a wall, a ground surface and a clinician's body while the syringe assembly is manipulated and moved during an injection procedure.

8. The injection system of claim 1, wherein the motion transfer cable comprises at least one protective outer sheath, wherein the inner core of the motion transfer cable is slidably movable relative to said at least one protective outer sheath.

9. The injection system of claim 1, wherein the syringe adapter of the syringe assembly is configured to receive syringes of varying sizes, shapes and types.

10. The injection system of claim 1, wherein the syringe adapter is configured to removably couple to a flange portion of the syringe.

11. The injection system of claim 1, wherein the injection system is in data communication with an imaging device configured to help locate a targeted anatomical location within the patient.

12. The injection system of claim 11, wherein the imaging device comprises an ultrasound device.

13. The injection system of claim 1, wherein the at least one motor assembly comprises a stepper motor.

14. The injection system of claim 1, further comprising a handpiece assembly, an interior portion of said handpiece being configured to receive and secure the syringe assembly.

15. A method of injecting a fluid stored within a pre-filled syringe into a patient, comprising:
securing a pre-filled syringe to a syringe adapter of a syringe assembly of an injection system;
wherein the injection system further comprises a motor assembly and a motion transfer cable, the motion transfer cable comprising an inner core, wherein the motion transfer cable mechanically couples the syringe assembly to the motor assembly;
coupling a first terminal of the motion transfer cable to an actuator of the motor assembly, wherein at least one motor of the motor assembly is configured to selectively move the actuator;
coupling the second terminal of the motion transfer cable to a plunger assembly of the syringe assembly, said plunger assembly being configured to selectively move within an interior of the pre-filled syringe in order to discharge fluid from an outlet of the pre-filled syringe;
moving the actuator using the motor assembly in response to an injection instruction, wherein moving the actuator causes a corresponding linear motion in the first and second terminals and the inner core of the motion transfer cable extending between said first and second terminals;
wherein movement of the second terminal toward the pre-filled syringe urges the plunger assembly into the interior of the pre-filled syringe and transfers a volume of fluid through the outlet of the pre-filled syringe;
wherein fluid exiting the outlet of the pre-filled syringe passes through a needle assembly removably secured to a distal end of the pre-filled syringe and into a target anatomical location of a patient; and
wherein the syringe assembly is configured to be grasped and manipulated by a clinician during an injection procedure.

16. The method of claim 15, wherein the injection system is configured to receive instructions for moving the actuator and transferring a volume of fluid out of the pre-filled syringe.

17. The method of claim 15, wherein the motor assembly is positioned within or coupled to a fluid delivery module.

18. The method of claim 15, wherein the motion transfer cable is generally flexible to permit the syringe assembly to be manipulated relative to the motor assembly by a clinician during an injection procedure.

19. The method of claim 15, wherein the motion transfer cable comprises at least one protective outer sheath, the inner core of the motion transfer cable being slidably movable relative to the at least one protective outer sheath.

20. The method of claim 15, wherein coupling the first terminal of the motion transfer cable to the actuator comprises securing the first terminal to a quick-connect coupling of the actuator.
